(19) United States
(12) Patent Application Publication
(10) Pub. No.: US 2012/0028822 A1
(43) Pub. Date: Feb. 2, 2012

(54) METHODS, FLOW CELLS AND SYSTEMS FOR SINGLE CELL ANALYSIS

(75) Inventors: Richard Joseph, Stoughton, MA (US); James Dimeo, Needham, MA (US); Mirna Jarosz, Arlington, MA (US); John F. Thompson, Warwick, RI (US); Jayson Bowers, Cambridge, MA (US); Scott Chouinard, Medford, MA (US); Philipp Kapranov, Sunnyvale, CA (US); William J. Efcavitch, San Carlos, CA (US); Christopher Hart, Cambridge, MA (US); Fatih Ozsolak, Boston, MA (US)

(73) Assignee: HELICOS BIOSCIENCES CORPORATION, Cambridge, MA (US)

(21) Appl. No.: 13/125,932

(22) PCT Filed: Nov. 4, 2009
(86) PCT No.: PCT/US09/63292

§ 371 Date: Aug. 19, 2011

Related U.S. Application Data
(60) Provisional application No. 61/111,128, filed on Nov. 4, 2008.
(60) Provisional application No. 61/111,004, filed on Nov. 4, 2008.

(51) Int. Cl. C40B 30/04; C40B 30/00; C12M 1/34; C12Q 1/68

(52) U.S. Cl. 506/9; 435/6.1; 506/7; 435/287.1

(57) ABSTRACT

A method, flow cell and/or device for increasing the recovery of a limiting analyte in a sample, e.g., for single molecule analysis is disclosed. Methods for preparing a nucleic acid sample from a single cell and capturing nucleic acids on a surface configured for use in or with single molecule analysis are also provided.

### CROSS-REFERENCE TO RELATED APPLICATIONS

This application claims priority to U.S. Patent Application Ser. Nos. 61/111,004 and 61/111,128, both of which were filed on Nov. 4, 2008, under 35 U.S.C. §119. The contents of the aforementioned applications are hereby incorporated by reference in their entirety.

### BACKGROUND

Conventional nucleotide sequencing is frequently accomplished through bulk techniques. Bulk sequencing techniques are typically not useful for the identification of subtle or rare nucleotide changes due to the many cloning, amplification and electrophoresis steps that complicate the process of gaining useful information regarding individual nucleotides. As such, research has evolved toward methods for rapid sequencing, such as single molecule sequencing technologies. However, effective diagnosis and management of important diseases through single molecule sequencing is impeded by lack of cost-effective tools and methods for screening individual molecules or cells. For example, many samples (e.g., human samples) used for single molecule sequencing and/or sequencing analysis for individual cells are available in limited quantities. Thus, maximum utilization of the limited sample amounts is highly desirable.

Therefore, a need exists for methods and devices that allow more effective sample utilization for nucleic acid sequencing of single molecules or cells.

### SUMMARY

The present invention provides, at least in part, methods, flow cell devices and/or systems for improving single molecule analysis and/or analysis of nucleic acids from an individual cell. In one aspect, the invention provides methods and flow cells for increasing the recovery of an analyte in a sample by providing a flow cell with a mechanism for sample recircularization, e.g., through a reactive vessel. In another aspect, the invention features methods for improving the analysis of nucleic acids from individual cells, e.g., by tracking the origin and/or source of a nucleic acid in a sample to a given cell or cell type. Thus, methods, flow cells and/or systems useful for improving sample processing and analysis are provided herein.

Accordingly, in one aspect, the invention features a method for increasing recovery of an analyte(s) in a sample, e.g., a nucleic acid sample, in a device (e.g., a flow cell or similar device). The method includes providing a mechanism for sample recircularization in the device or flow cell, e.g., using a reactive vessel, such that the analyte(s) in the sample has two or more, e.g., multiple, exposures to the reactive vessel, (e.g., a given functionalized reactive vessel).

In one embodiment, a method for loading a flow cell or similar device for single molecule analysis is provided. The method includes: loading the sample in a flow cell or device that includes at least one inlet port and at least one outlet port, wherein each of the inlet and outlet ports is coupled to at least one loading block (e.g., a microfabricated loading blocks); and wherein the loading blocks are constructed and/or arranged (e.g., joined) so as to permit sample loading and/or fluid recirculation, e.g., through at least one reaction vessel. In certain embodiments, the flow cell or device contains more than one reaction vessel.

In another embodiment, a method for facilitating loading of a flow cell or similar device is provided. The method includes providing at least a first and a second loading block comprising a fluidic coupling therebetween, in which at least one of the first and second loading blocks is constructed and/or arranged to couple to the flow cell. In certain embodiments, the loading block provides fluid to the flow cell.

In another aspect, the invention features a flow cell or device that includes at least one inlet port and at least one outlet port, wherein each of the inlet and outlet ports is coupled to at least one loading block (e.g., a microfabricated loading block); and wherein the loading blocks (e.g., the microfabricated loading blocks) are constructed and/or arranged (e.g., joined) so as to permit sample loading and/or fluid recirculation (e.g., through at least one reaction vessel). In certain embodiments, the flow cell or device contains more than one reaction vessel.

Embodiments of the aforesaid methods and flow cells may include one or more of the following features.

In one embodiment, the loading blocks of the flow cells individually access each of the reaction vessels. In another embodiment, the joining is by means of glass capillaries.

In one embodiment, after introducing the sample, the system is closed permitting recirculation (e.g., as a result of temperature or electrical gradients) of the sample repeatedly through a reaction vessel. For example, the reaction vessel can be a channel or a microfabricated vessel. The individual channels and/or microfabricated vessels can be accessed by the inlet port and outlet port. In another embodiment, the reaction vessel includes a means of maintaining sample agitation, e.g., by means of magnetic beads, or back-forth fluid flow control. In yet another embodiment, the reaction vessel has a single inlet and outlet port, and multiple reaction locations are defined by analytes (e.g., haptens, antibodies, or nucleic acid) specifically attached in defined locations. For example, the analytes are applied to defined locations by spotting (e.g., mechanical, inlet spraying, or sonic), or synthesized at defined locations. In one embodiment, the loading blocks prevent, minimize or otherwise reduce sample intermixing during circulation.

In one embodiment, the loading of the flow cell includes drawing the sample directly from a multi-well device (e.g., a microplate). In another embodiment, the samples are applied to each of the reaction vessels prior to attaching the recirculating system.

In another aspect, the invention features a method for sequencing analysis of nucleic acid from individual cells. The method includes: (i) selecting individual cells; (ii) lysing cells; (iii) capturing nucleic acids on surface; (iv) adding a universal sequence; and (v) sequencing at least a portion of the nucleic acid. In certain embodiments, the order of steps (iii) and (iv) can be reversed.

In one embodiment, the nucleic acids in the individual cells are barcoded. For example, the barcoding is via viral vectors, transposons, and/or a spatial:temporal association (e.g., a spatial:temporal association derived from sorting (e.g., via fluorescence activated cell sorter (FACS) and/or specific antibody capture) and maintained using a surface (e.g., a surface comprising wells (e.g., a microplate)). In another embodiment, methods of direct mechanical spotting, inkjet spraying, or sonic spraying can be used to apply the cells to the surface.

In yet another embodiment, the cells described herein are red blood cells. The red blood cells can be lysed, for example, with hemolysin.

In one embodiment, the nucleic acids described herein are fragmented prior to being captured on a surface. For example, the fragmentation is enzymatic (e.g., by a restriction enzyme and/or nuclease), heat induced, chemical, and/or physical stress (e.g., sound waves and/or osmotic pressure). In one embodiment, the nucleic acid is DNA. In another embodiment, the nucleic acid is RNA. For example, the RNA fragments can be treated with periodate to produce 3′-ends of RNA with aldehyde moieties and captured on a surface with reactive amines for a Schiff base. The surface can be further treated to reduce the Schiff base.

In one embodiment, the universal sequence described herein is added by one or more of: ligation; a single dNTP and terminal deoxynucleotidyl transferase; or a single ATP and polyA polymerase. In another embodiment, the universal sequence is added during a polymerase mediated copying of the nucleic acid. For example, the copying can additionally add a functional group (e.g., an amine or a phosphate group) so as to enable ligation to surface anchored oligonucleotides onto the copied fragments. This example enables the chemical attachment of the copied samples to a surface (e.g., a surface containing epoxides).

In one embodiment, the surface described herein is a bead, or a planar or three dimensional surface. In another embodiment, the surface is glass or silicon (e.g., having an epoxide coating). In yet another embodiment, the surface is coated with capture oligonucleotides. For example, the capture oligonucleotides are 20-50 bases in length. The capture oligonucleotides can comprise all possible combinations of the sequences found in the sample nucleic acid. The capture oligonucleotide can also have a sequence complementary to the universal primer, e.g., anchored to the support via the 5′-end. In one embodiment, the surface is coated with the capture oligonucleotides at a density of greater than 10, 20, 30, 40, 50, 60, 70, 80, 90, 100, 110, 120, 130, 140, 150, 200 or more objects per μm².

In another embodiment, the sequencing is performed by synthesis, ligation, and/or hybridization. For example, the multiple rounds of hybridization, detection, and/or denaturing can be performed, each round using different interrogation oligonucleotides. In some embodiments, the sequencing is on individual, optically resolvable molecules. In other embodiments, the nucleic acid on the surface is amplified prior to sequencing, while in others it is not.

In one embodiment, a carrier nucleic acid is added to the sample nucleic acid. For example, the carrier nucleic acid is not able to hybridize to the capture oligonucleotides on the surface, and/or the carrier nucleic acid is modified in a way (e.g., modified with uracil residues and degraded using USER enzyme; modified to comprise a sequence of bases unique to the carrier; or modified with one member of a binding pair (e.g., biotin and streptavidin)) such that it can be selectively removed and/or degraded. For example, the carrier nucleic acid is modified by hybridization to a support modified with a complement of the sequence unique to the carrier.

All publications, patent applications, patents, and other references mentioned herein are incorporated by reference in their entirety.

Other features, objects, and advantages of the invention will be apparent from the description and drawings, and from the claims.

### BRIEF DESCRIPTION OF THE DRAWINGS

FIG. 1 shows an example of one configuration of a flow cell wherein 2 loading blocks are utilized to address the inlet and outlets joined by a fluidic connection to enable sample recirculation.

FIG. 2 shows an example of another configuration wherein the loading blocks are microfabricated so as the internally provided fluidic connections for input of the sample and for recirculation.

FIG. 3 is a schematic diagram of two imaging areas, A and B, each with three spots, where the two imaging areas are separate flow cells attached to the same holder;

FIG. 4 is a schematic diagram of two imaging areas, A and B, where the surface of a single flow cell is divided into separate areas that have separate fluidic connections;

FIG. 5A shows a schematic diagram of an embodiment of a flow cell that has multiple channels;

FIG. 5B shows a schematic of parallel hydrophobic (hatched) and hydrophilic (open) channels;

FIG. 5C shows a schematic of a flow cell having channels created by hydrophobic and hydrophilic regions;

FIG. 6 depicts an approximation of spots in circular annuli;

FIG. 7 is a schematic top view of an exemplary embodiment of a flow cell;

FIG. 8A is an exploded perspective view of a first embodiment of the flow cell of FIG. 7 showing top and side surfaces of the components that comprise this first embodiment of the flow cell;

FIG. 8B is an exploded perspective view of the of the flow cell of FIG. 8A but showing bottom and side surfaces of the components;

FIG. 8C is a perspective view like FIG. 8B but with two substrates assembled;

FIG. 9A is an exploded perspective view of a second embodiment of the flow cell of FIG. 7 showing top and side surfaces of the components that comprise this second embodiment of the flow cell;

FIG. 9B is an exploded perspective of the flow cell of FIG. 9A but showing bottom and side surfaces of the components;

FIG. 9C is a perspective view like FIG. 9B but with the adhesive film disposed on the first substrate;

FIG. 9D is a perspective view of a fully assembled flow cell of FIGS. 9A-9C;

FIG. 10A is a perspective view according to an exemplary embodiment of a fully assembled flow cell;

FIG. 10B is a perspective view of the flow cell of FIG. 10A with a loading block being moved into position;

FIG. 10C is a perspective bottom view of the flow cell of FIG. 10B;

FIG. 10D is a perspective view according to an exemplary embodiment of a loading block;

FIG. 10E is a perspective view of the flow cell of FIG. 10B after the loading block has been moved into position;

FIG. 10F is a perspective view of the flow cell of FIG. 10E with an unloading block being moved into position;

FIG. 10G is a perspective bottom view of the flow cell of FIG. 10F;

FIG. 10H is a perspective view according to an exemplary embodiment of the flow cell shown in FIG. 10F after the unloading block has been moved into position;

FIG. 10I is a perspective view of the flow cell of FIG. 10H after the loading block and unloading block have been removed after loading;

FIG. 11 is a schematic view of an apparatus that can be used to perform analytical experimentation with an exemplary embodiment of the flow cell shown in FIG. 10H;

FIG. 12A is a perspective view of an inverted flow cell being placed into a flow chuck;

FIG. 12B is a perspective bottom view of the flow cell shown in FIG. 12A;

FIG. 12C is a perspective view of the flow cell shown in FIG. 12A after it has been placed in the flow chuck;

FIG. 13A is a perspective view of an inverted flow cell being placed into a flow chuck;

FIG. 13B is a perspective bottom view of the flow cell shown in FIG. 13A;

FIG. 13C is a perspective view of the flow cell shown in FIG. 13A after it has been placed in the flow chuck;

FIG. 14 is a perspective view of a dual flow cell assembly;

FIG. 15 is a schematic of an imaging area having multiple spots of biochemical molecules attached thereto;

FIG. 16 is an exemplary schematic showing molecules viewed as an image stack;

FIG. 17 shows an exemplary imaging system of the present invention;

FIG. 18A is a chart showing an estimation of the number of targets per flow cell versus sequence length of the target nucleic acid of interest; and

FIG. 18B is a chart showing an estimation of the spot diameter versus kilobases of target nucleic acid of interest.

### DETAILED DESCRIPTION

Certain embodiments described herein are directed to methods for improving overall utilization of rare samples when applying to a flow cell device for analysis. As many samples can only be obtained in limiting quantity, as in samples for analysis of human samples, devices which provide for maximum utilization are desired. This is especially the case when performing molecular analysis as when performing nucleic acid sequencing utilizing methods of single molecule analysis.

Apparatus(s) are currently able to be designed and implemented using either micro machining and assembly or microfabrication which are able to define extremely small sample volume reaction chambers from picoliter to microliter volumes. These reaction chambers can take on many different formats such as channels, cylindrical, or spherical vessels. In certain cases, individual reaction vessels can be defined as unique reaction sites on an otherwise uniform surface. These reaction sites might be defined by applying an analyte of interest at predefined locations on a surface. These analytes might also be synthesized directly at predefined locations.

In certain instances, sample volumes can only be reduced to a volume limited by a user's ability to physically handle such sample. Other issues of reducing sample volume have to do with limits in the ability to concentrate analytes. When trying to apply a sample of interest into a reaction vessel there are several current methods: i) flow the sample through so as to expose the sample to the reaction vessel on a single pass, ii) flow through the sample in a single pass in stages wherein one or more stage might represent an incubation for a period of time, or iii) make the reaction vessel sized appropriately to hold the entire sample. In methods i) and ii) the sample analyte gets one time equivalent exposure to reaction vessel before being disposed. In method iii) increasing the reaction vessel size is counter productive to high throughput low cost analysis. None of these methods make optimum usage of all the analyte available in the entire sample.

Certain features, aspects, embodiments and examples described herein address the problem of maximizing recovery of analyte in a sample for analysis in a flow cell device. One embodiment is to incorporate mechanisms for sample recirculation through the reactive vessel such that analyte(s) in the sample have multiple exposure to a given functionalized reaction vessel: an example of such being a reaction vessel with oligonucleotides attached and analyte in a sample being nucleic acid, either DNA or RNA. Hybridization capture of the nucleic acid in the sample onto the surface bound oligonucleotides generally is controlled by incubation time, temperature and dissolved salt to control stringency. Passing an analyte sample in which the volume is greater than the reaction vessel through multiple passes increases the chances of capturing the entirety of the analyte from the sample volume. Recirculation methods enable multiple passes of a given analyte to have exposure to the reactive surface in the vessel.

Certain embodiments described herein are directed to methods for improving analysis of nucleic acids from an individual cell. As many samples can only be obtained in limited quantity, as in samples for analysis of human samples, methods and devices which provide for maximum utilization are desired. This is especially the case when performing molecular analysis as when performing nucleic acid sequencing utilizing methods of single molecule analysis.

Certain features, aspects, embodiments and examples described herein address the problem of handling and recovery of analyte, e.g. nucleic acids such as DNA and/or RNA, in a sample for analysis in a flow cell device. One embodiment is to incorporate mechanisms for sample encoding so as to allow data tracking back to the individual sample. Sample encoding also allows samples from various sources to be mixed prior to analysis.

Analysis of nucleic acids from individual cells would encompass being able to isolate and manipulate individual cells. Additionally, it would be desirable to track through the process the origin of the nucleic acid to specific cells or cell types. Examples of tracking would include addition of markers (e.g., barcodes) into the nucleic acid of any given cell. Examples of how to barcode nucleic acid would be via use of viral vectors or transposons which integrate into host nucleic acid and leave their molecule signature. The signature might be the unique sequence of the virus or transposon or of an additional unique sequence input into the virus or transposon prior to exposing cells. Additional examples, depending upon method of cell manipulation might include arranging of individual cells on a surface. The cells are then tracked by the spatial arrangement and/or time of spotting. The spatial:temporal relationship is important in order to associate data about a cell if such cell(s) were characterized and isolated via a method such as fluorescence flow cell sorting (FACS).

Individual cells can also be isolated specifically using various specific capture methods. For example, a microplate can be coated with one or more types of antibodies direct at cellular targets located on the outside of the cell membrane or cell wall. Another example is use of lectins to sort cells via their cell surface carbohydrate compositions. The antibodies or lectins can be attached to planar surfaces by various means such as contact printing, inkjet spraying, or sonic spraying.

Following isolation of individual cells, the cell can be lysed, or the cellular membrane may otherwise be disrupted, to release the nucleic acid contained within. The lysing of the cells might use detergents, enzymes, physical mechanisms (sonic or hydrostatic pressure), or combinations. Depending upon cell type, for example if using red blood cells, specific enzymes such as hemolysin are known which specifically lyse these cells. Depending upon whether the analysis is for DNA or RNA, enzymes such as RNase or DNase might additionally be utilized. The analysis of DNA is performed for example to determine genomic structure which might include mutations (SNPs, insertions, deletions), gross chromosomal rearrangements, and chromosomal or gene copy number variations. The analysis of RNA is performed for example to determine relative gene activity, gene: structure function relationships, alternative transcription start sites and or alternative splice sites during process of RNA to mRNA.

For many applications which perform nucleic acid analysis, for example nucleic acid sequencing, it is desirable to fragment the intact chromosomal DNA or RNA in a cell. Fragmentation of nucleic acid can be by a variety of methods, for example enzymatic using restriction enzymes or nucleases or physical shearing using for example sonication. Additionally, attachment of unique sequence of nucleic acid to the fragments is desirable. The unique sequence may be a universal sequence in that the same sequence attached to all fragments. The sequence might be unique sequence comprised of a mixture of all 4 nucleoside bases or a homopolymer of a single nucleoside base. The sequence can be added either by ligation or enzymatic synthesis. The preferred enzymes for ligation are: a DNA ligase, RNA ligase or CircLigase which has been shown to join single strands together, and preferred enzymes for addition of a homopolymeric sequence are: terminal deoxynucleotidyl transferase (TdT) or polyA polymerase.

The universal sequence, depending upon when attached to the fragments, can function as a means to attach fragments to a surface, for example the surface is coated with a reverse complement to the universal sequence, or as a sequencing primer in order to initiate sequencing by synthesis analysis. In some examples the universal sequence can function as both surface capture and sequencing primers, especially when an oligonucleotide containing a complementary sequence to the universal sequence is attached to the surface via the 5′-end. Optionally, the sample nucleic acid fragments can be attached to a surface via hybridization capture wherein the surface is coated with random sequence oligonucleotides representing all possible sequences expected to found in the sample. The universal sequence region for priming sequencing by synthesis can be added following binding to the surface. Some methods for sequencing analysis, for example sequencing by hybridization, may or may not require addition of a universal primer for sequencing however the universal primer may aide surface capture of fragmented nucleic acids.

When analyzing RNA from a cell it is possible to fragment RNA using exposure to heat and magnesium ions. Following RNA fragmentation by any means one embodiment is to chemically modify the 3′-ends of the fragments to enable surface attachment in lieu of attaching a universal sequence for this purpose. RNA has cis diols on the 2′ and 3′ ribose of the 3′ terminal base. Treatment of this RNA with periodate cleaves the cis diols leaving aldehyde moieties on the 2′ and 3′ sugar positions. Exposure of RNA treated in this manner to a surface which has primary amines results in forming a direct chemical attachment to the surface via a Schiff's base. The Schiff's base can be further reduced to stabilize the attachment.

When analyzing RNA or DNA either before or after fragmentation, it is possible to make a primer dependent, polymerase mediated copy. One example of such a process would be to use a primer dependent polymerase wherein the primer has from 6-10 random bases on the 3′ end and the universal sequence (20-50 bases). In another example, the 5′ end of the primers might include a functional group, e.g. amine or aldehyde or phosphate, which would permit direct chemical attachment to the surface, e.g. amine or aldehyde addition to epoxides on surface, or ligation, e.g. 5′-phosphate fragments to surface attached oligonucleotides 3′-OH.

Six major high-throughput sequencing platforms are currently available: the Genome Sequencers from Roche/454 Life Sciences (Margulies et al. (2005) Nature, 437:376-380; U.S. Pat. Nos. 6,274,320; 6,258,568; 6,210,891), the 1G Analyzer from IIlumina/Solexa (Bennett et al. (2005) Pharmacogenomics, 6:373-382), the SOliD system from Applied Biosystems (solid.appliedbiosystems.com), the Heliscope™ Sequencer from Helicos Biosciences (see, e.g., U.S. Patent App. Pub. No. 2007/0070349 and www.helicosbio.com), sequencing service provided by Complete Genomics (http://www.completegenomicsinc.com/technology/technicalDetails.aspx), and GeneChip® arrays from Affymetrix (www.affymetrix.com). The various methods used to obtain sequencing information from each of these platforms employ different mechanisms categorized as either i) sequencing by synthesis (template dependent, polymerase addition of nucleotides), ii) sequencing by ligation (template dependent, ligase coupling of oligonucleotides), or iii) sequencing by hybridization (enzyme-free oligonucleotide:oligonucleotide hybridization).

Certain features also include flow cells incorporating additional features in design or function that enable handling and capture of nucleic acids on a surface. One example would be the coating of surfaces with capture oligonucleotides at much higher density, e.g. 10× or 100× or higher, than is the standard in single molecule applications generally defined as objects spaced farther apart than the diffraction limit of light used in the analytical system so as to drive the hybridization capture reaction. Hybridization capture of the nucleic acid in the sample onto the surface bound oligonucleotides generally is controlled by incubation time, temperature and dissolved salt to control stringency. Another example would be passing an analyte sample in which the volume is greater than the reaction vessel through multiple passes increasing the chances of capturing the entirety of the analyte from the sample volume. Recirculation methods enable multiple passes of a given analyte to have exposure to the reactive surface in the vessel.

When handling small quantities of analyte, nonspecific adsorption to surfaces can be an issue which is in some instances overcome by adding a carrier substance to the sample. Generally the carrier is of similar composition as to the sample, for example adding exogenous DNA (or synthetic oligonucleotides) to DNA sample or RNA to an RNA sample. The carrier substance might include various modifications which enable removal of the carrier from the sample, for example modified with one member of a binding pair, e.g., biotin or a different universal sequence, so as to permit removal by exposure to a support labeled with either streptavidin or an oligonucleotide complementary to the different universal sequence found only on the carrier. Optionally, the carrier substance may lack a feature found on or added to the sample analyte, for example the might carrier lack the universal sequence added to the nucleic acid fragments from the sample to enable surface capture by hybridization and/or priming of the sequence reaction.

In one example applicable to the methods and flow cell devices described herein, efficient determination of analytes will be desirable when performing single molecule analysis. When analytes are present in extremely dilute solutions and/or occur rarely in a given sample volume, such as one molecule, it is important that such sample be conserved and maximize the odds that the one molecule will be detected. Should a sample be passed through a reaction vessel in a single pass and such molecule has not had sufficient time to react, detection of such molecule will not occur.

In some examples, a method for loading a flow cell for single molecule analysis, might include a flow cell which contains inlet and outlet ports; the flow cell contains more than one reaction vessel for analysis of different samples or analytes within a sample. A loading block device can be coupled to each of the inlet and outlet ports, e.g., fluidically coupled, and the loading blocks may be joined so as to permit fluid recirculation. The loading blocks in one aspect are joined via the reaction vessel and, optionally, additionally through a tubing, capillary or fluid conduit joining one block to the other thus closing the loop or fluid circuit

In some examples, the loading blocks individually access each of the reaction vessels. Other examples include a loading block which is microfabricated to include individual access to all reaction vessels. In certain configurations, the loading blocks may be constructed and arranged to prevent sample intermixing during circulation.

The reaction vessel can be of various shapes one of which is a channel. The inlet and outlet port can be located at the distal ends of a channel and access individual channels. The reaction vessel may also be microfabricated into various shapes wherein the inlet and outlet port access individual reaction vessels.

In another example, after introducing sample the system is closed permitting recirculation of the sample repeatedly through a reaction vessel. The recirculation of fluid may be the result of temperature or electrical gradients.

In other examples, loading of the flow cell includes drawing sample directly from a multi-well device. The multi-well device might have been used in sample preparation or isolation in preparation for analysis in the flow cell. In preferred examples, the multi-well device has a similar number of wells as the flow cell has reaction vessels.

In other examples, the reaction vessel includes a means of maintaining sample agitation. One example of agitation is by means of magnetic beads. Another example is by means of controlling the fluid flow so as to pass the fluid back-forth over a given surface several times.

In other examples, a single reaction vessel with a single inlet and outlet can include multiple reaction locations which are defined by analytes specifically attached in defined locations. The analytes might be the same or different from location to location. In some examples, the analytes are applied to defined locations by spotting. The spotting used might be mechanical, by spraying, by ink-jet deposition, inlet spraying, or sonic deposition. The analytes can also be synthesized at defined locations. For example, the analytes can be haptens, ligands, antibodies, or nucleic acid. In some examples, hapten surfaces are used to detect antibody analytes in a sample. In other examples, ligands are used to detect receptors in a sample. In other examples, antibodies are used to detect haptens or proteins in a sample. In other examples, nucleic acids in a reaction vessel can be used to detect presence or absence of a gene (DNA) or gene product (mRNA), copy number of a gene, quantitate expression level of a gene, detect mutations, and provide sequence information. In a specific example, the flow cell reaction vessels might be used in methods of single molecule sequencing based upon either sequencing by synthesis, sequencing by ligation, or sequencing by hybridization.

### Exemplary Flow Cells, and Materials and Methods for Producing the Same

Certain examples of the flow cells, and materials and methods used to produce them are described in more detail below. In some examples, the flow cell may include one or more imaging surfaces that include a suitable material, e.g., a plastic such as PEEK or other suitable material, to permit imaging and/or analysis. In on embodiment, a surface for the imaging areas of a flow cells can be an epoxide surface on a glass or fused silica slide or cover slip. For example, the surface can be about a 10 mm to about a 100 mm round cover glass. The surface can have a thickness of about 0.05 mm to about 0.45 mm. In some embodiments, the cover glass is a 40 mm round cover glass (Erie Scientific) and has a thickness of 0.15 mm. The imagable area of the cover glass can be from about 10 mm² to about 10,000 mm². In one embodiment, the imagable area of the cover glass is about 690 mm², which may be split amongst the imaging areas of the flow cell. Where the flow cell comprises two imaging areas, each imaging area can be, for example, about 345 mm².

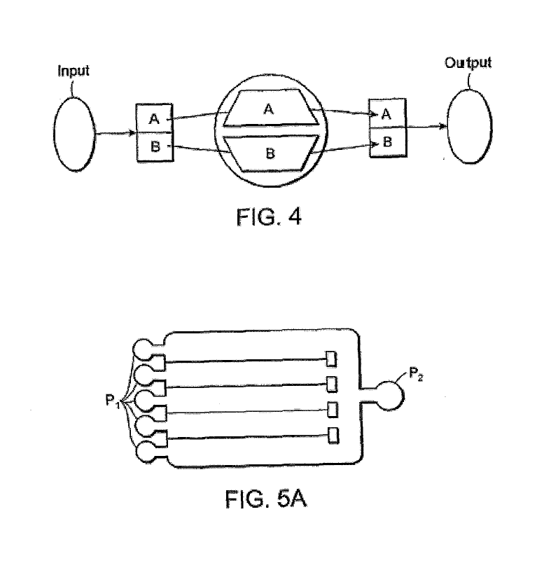

In certain examples, the imaging areas can be part of separate flow cells that are attached to the same holder and mounted together into the interrogation device (such as a microscope). In another embodiment, the imaging areas are part of a single flow cell and each imaging area can be surrounded by a gasket or other material that isolates the imaging areas from each other as described above. FIG. 3 is a schematic diagram of two imaging areas, A and B, each with three spots, where the two imaging areas are separate flow cells attached to the same holder. FIG. 4 is a schematic diagram of two imaging areas, A and B, where the surface of a single flow cell is divided into separate areas that have separate fluidic connections. Additional configurations of one or more flow cells that include two or more imaging areas will be recognized by the person of ordinary skill in the art, given the benefit of this disclosure.

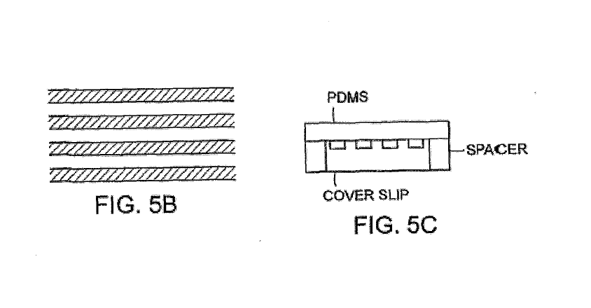

Referring to FIG. 5A, one embodiment of a flow cell can include a series of two or more fluid conduits, e.g., flow paths also referred to herein in certain instances as channels. Each channel can have a separate fluid inlet Pj or may include split or multiple fluid inlets that converge at one or more portions of the fluid conduit. The channels can have separate fluid outlets or can share a common fluid outlet P2. The channels can be formed by masking. For example, parallel hydrophobic and hydrophilic regions can be created, as shown in FIG. 5B. Hydrophobic and hydrophilic regions can be formed by using a glass cover slip and a polydimethylsiloxane (PDMS) slide as shown in FIG. 5C. The PDMS surface can be made selectively hydrophobic by masking and exposing to plasma.

In certain examples, the reagents used for performing the reaction component of the biochemical assay can be introduced simultaneously. Because the fluid flows in each channel usually have very low Reynolds Numbers (″1), and typically have the same viscosity, applying constant pressure down the channels provides multiple parallel fluid flow paths. The application of pressure down the channels can be accomplished by applying pressure to the inlet, applying suction to the outlet, or a combination of both to achieve a suitable flow rate through the channels. Such pressure may be continuous, pulsed, intermittent or applied or introduced using other suitable techniques. Where the biochemical assay involves nucleic acids, nucleic acids can be attached to the channels as described above. Multiple different oligonucleotides can then be added to the channels and hybridized to the attached nucleic acids in a single step. In addition, depending on the ratio of the imaging time compared to the reaction time as described above, the channels can be divided into two or more groups such that one group of channels is subjected to the reaction component of the biochemical assay, while the other group of channels is interrogated.

In some examples, the cover glass can include a guard band of about 2 mm, or the edges of the cover glass can be sloped so that the interrogation device does not interfere with the imaging areas. In one embodiment, the interrogation device is a Nikon Plan APO TIRF 60×/1.45 objective. Other suitable interrogation or detection devices are described below.

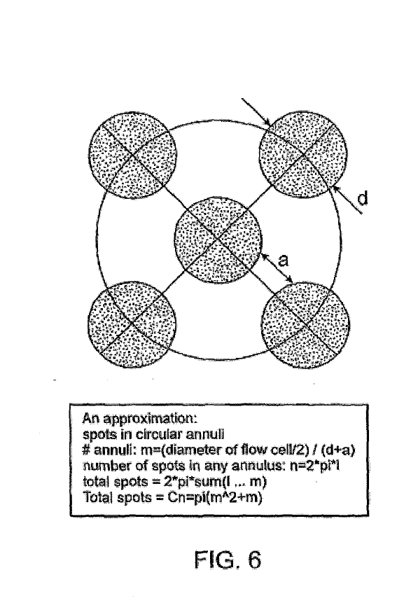

As described herein, embodiments of flow cells having minimal volume provide several advantages. For example, the volume of the flow cell can be from about 1 to about 1000 microliters. Furthermore, the exchange of internal volume of the flow cells is rapid. In one embodiment, the exchange takes less than 1 second at 3000 kPa driving pressure and the maximum Reynolds Number at 4 degrees Celsius is less than 1. The bow of the cover slip is typically less than 20% of initial channel height during pumping. FIG. 6 depicts one illustration of spots in circular annuli.

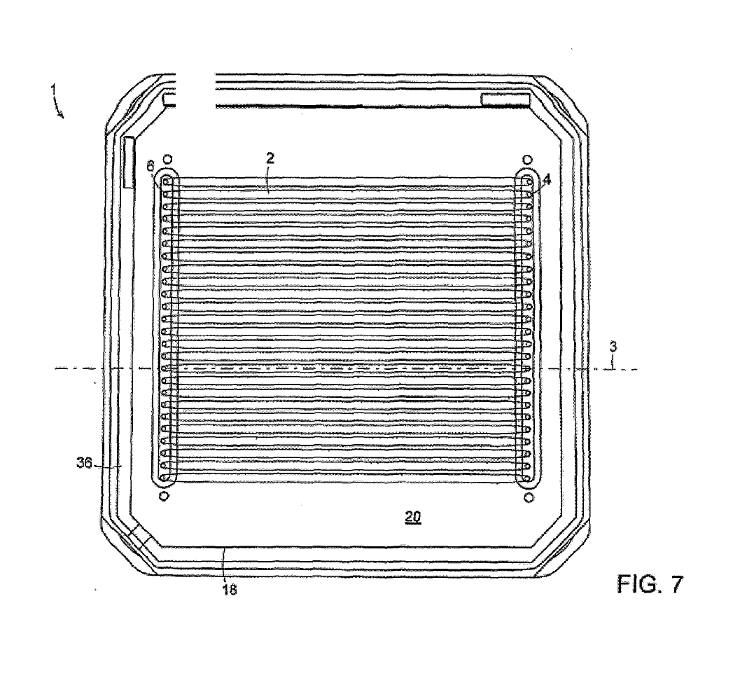

In another embodiment, a multi-channel flow cell for handling and analyzing microfluidic volumes and related biological materials is designated 1 in FIG. 7. The multichannel flow cell can be used in a wide variety of applications such as, for example, performing single molecule sequencing.

The flow cell 1 includes a plurality of channels 2 oriented substantially parallel to each other. Each channel 2 has an inlet 4 for loading a fluid into the channel 2, and an outlet 6 for removing fluid from the channel 2. The channels 2 each have a capacity, for example, of about 3 microliters to about 15 microliters. Each of the channels 2 extends longitudinally along an axis 3 from one of the inlets 4 to a corresponding one of the outlets 6. As shown, the channels 2 can have a uniform width throughout the axis 3, however they may be tapered or curved in width and/or in depth depending on the desired application of the flow cell 1. Also, multiple channels 2 are shown but, of course, the flow cell 1 can have just a single one of the channels 2.

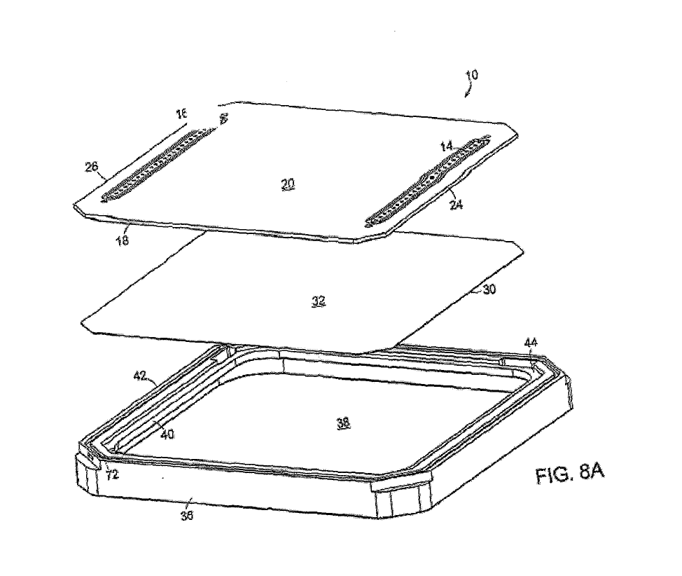

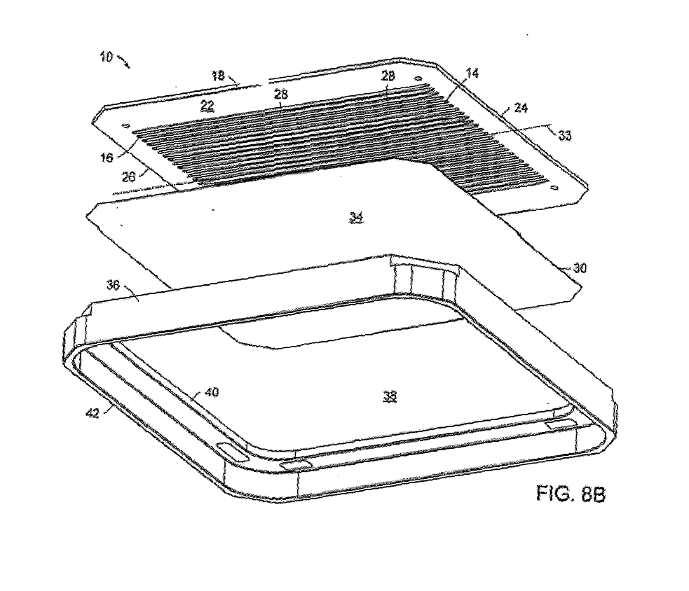

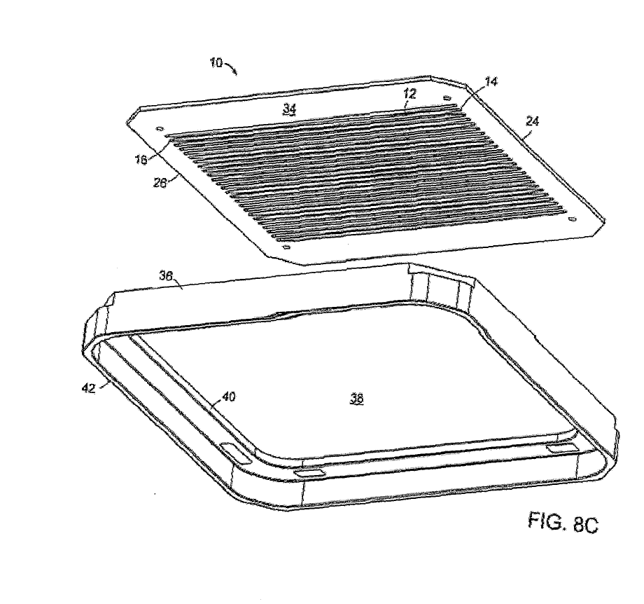

Referring now to FIGS. 8A-8C, individual components of a first embodiment of a flow cell 10 are shown prior to assembly. This illustration of a flow cell 10 includes a first substrate 18 having a first surface 20, e.g., a top surface, (FIG. 8A) and a second surface, e.g., a bottom surface 22 (FIG. 8B), opposite the first surface 20. The first substrate 18 further includes a plurality of inlet apertures, holes or ports 14 and a plurality of outlet apertures, holes or ports 16 formed therein, and each of these apertures, holes or ports 14, 16 extends through the substrate from the top surface 20 of the first substrate 18 to its bottom surface 22. The inlet holes 14 are aligned in a row near one edge 24 of the first substrate 18, and the outlet holes 16 are aligned in a row near an opposing edge 26 of the first substrate 18.

Referring in particular to FIG. 8B, each inlet hole 14 can include a corresponding or matching outlet hole 16. A plurality of recesses 28 can be etched, carved, molded, machined into or otherwise be present in the bottom surface 22 of the first substrate 18 along an axis 33 extending from each inlet hole 14 to its corresponding outlet hole 16. The first substrate 18 may be formed to any desired depth or width needed to form the desired channel 12 size and shape. In one embodiment, the size of the channels is chosen to make sure that the flow remains laminar at the desired flow rate.

In certain examples, the flow cell 10 may further include a second substrate 30 having a top surface 32 (FIG. 8A) and a bottom surface 34 (FIG. 8B). The second substrate 30 is for assembly to the first substrate 18, such that the top surface 32 of the second substrate 30 contacts the bottom surface 20 of the first substrate 18. Once the two substrates 18, 30 have been secured to each other, the recesses 28 are sealed and form channels 12 (FIG. 8C). In alternative embodiments, the recesses 28 can be etched, carved, molded, machined or otherwise introduced into the top surface 32 of the second substrate 30. The first substrate 18 can be selectively attached to the second substrate 30 by use of a variety of mechanical fasteners or by use of any of a variety of adhesives applied to the top surface 32 and/or the bottom surface 22. In other embodiments, melt soldering, melting, laser welding and the like may be used to attach the surfaces. Additional materials and methods for attaching the top and bottom surfaces will be readily selected by the person of ordinary skill in the art, given the benefit of this disclosure.

The first substrate 18 and the second substrate 30 each can be manufactured from any of a variety of materials or combinations of materials as long as the substrates 18, 30 are compatible with the microliter volumes passed therethrough, and to which any substances in the microliter volumes will not stick. The surfaces 22, 32 can also be passivated so that samples, such as DNA, only adhere to the desired surface. Passivation reagents include, for example, amines, phosphate, water, sulfates, detergents, bovine serum albumin (BSA), human serum albumin (HSA), glycols such as polyethylene glycol (PEG) or polymers such as POP-6(R) sold by Applied Biosystems.

The substrates 18, 30 are generally formed of a material that will allow light and/or energy of appropriate wavelength(s) to pass therethrough. This is because light of one or more wavelengths is passed through the substrates 18, 30 to illuminate the material(s) within the channels 12, in one use of the flow cell 10. The substrates 18, 30 can be glass, fused silica, sapphire, polydimethylsiloxane (PDMS), polymethyl methacrylate (PMMA), polyetheretherketone (PEEK), a suitable clear plastic such as acrylic or polycarbonate or combinations thereof. In some embodiments, the materials used as substrates 18, 30 can be the same or different, and adhesives and/or mechanical fasteners are not necessary to secure the substrates 18, 30 to each other but instead the substrates can be attached or otherwise mated using suitable welding, soldering or etching techniques.

In some examples, the flow cell 10 further includes a frame 36. The frame 36 includes an inside edge 40 and an outside edge 42, and the frame 36 defines a rectangular shaped opening 38. The frame 36 further includes a recess 44 (FIG. 8A) formed along the inside edge 40 for receiving and selectively securing (with, for example, an adhesive) the assembled substrates 18, 30 to the frame 36. The frame 36 can be constructed of any material with thermal expansion characteristics similar to those of the two substrates 18, 30, such as, for example, glass filled polycarbonate or multiple composite plastic. Alternatively, if there is a thermal mis-match, a highly-flexible glue, such as silicone, can be used. In certain examples, the coefficient of thermal expansion of the frame and the substrates may vary up to about 5-10% in the use environment conditions and still provide good fluid flow without undue mechanical stress on the overall device.

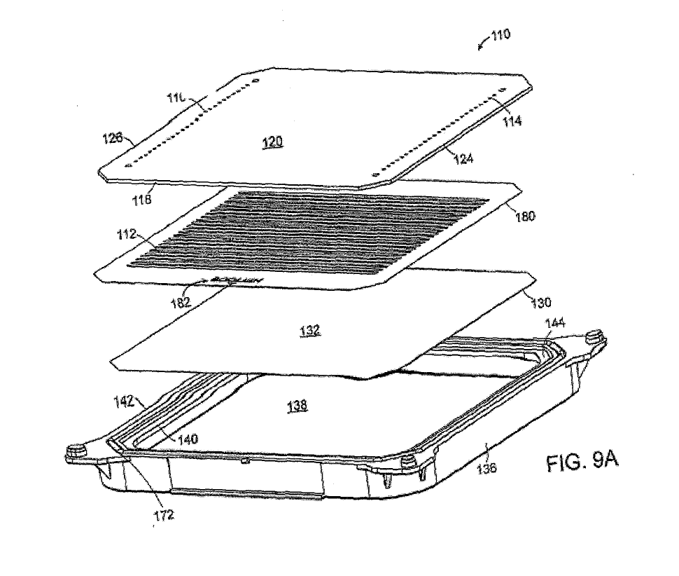

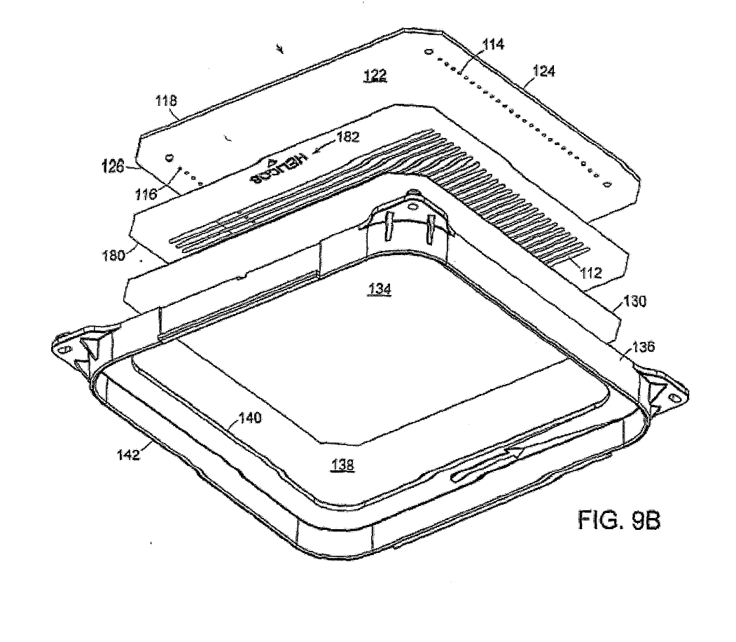

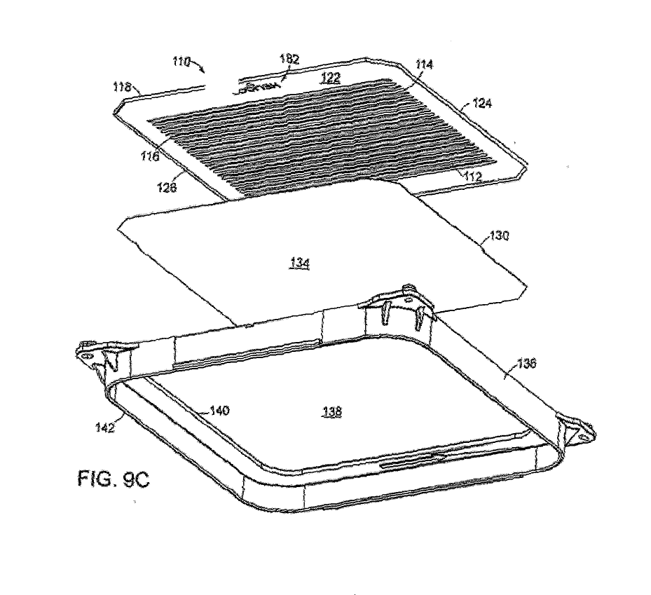

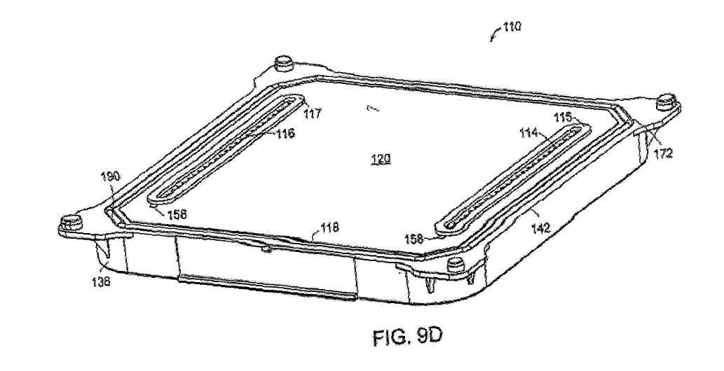

Referring now to FIGS. 9A-9D, another illustrative embodiment of a flow cell 110 according includes channels 112 formed in a manner different than described above with reference to FIGS. 8A-8C. For example, an adhesive can be disposed on a film or a delivery substrate in a desired bonding pattern. The film or delivery substrate can then be used to apply the patterned adhesive to one surface of a material to be bonded and then the film is pealed away and discarded leaving behind the adhesive disposed on the surface only in the desired locations and in the desired pattern. A second material can then be placed in contact with the first material to bond the two materials. Micronics, Inc. of Redmond, Wash. is one supplier of this technology. The film or delivery substrate may include a release agent or other suitable materials to facilitate transfer of the adhesive from the film or delivery substrate to the desired material.

In certain embodiments, the flow cell 110 can include a first substrate 118 having a first surface 120, e.g., a top surface, and a second surface 122, e.g., a bottom surface, opposite the first surface 110. The first substrate 118 may further include a plurality of inlet apertures, ports or holes 114 and a plurality of outlet apertures, ports or holes 116. A pattern of adhesive can disposed on a piece of film or a delivery substrate 180, which may be a film or take other forms such as, for example, a block, a substrate having a scrim or the like, in a predetermined bonding pattern. Using the technique described above, the film 180 can be used to apply the adhesive pattern to the bottom surface 122 of the first substrate 118 (FIG. 9C).

In certain examples, the flow cell 110 further includes a second substrate 130 having a first surface 132. e.g., top surface, and a second surface 134, e.g., a bottom surface. As an alternative to applying the adhesive to the bottom surface 122 of the first substrate 118 as described above, the adhesive may be disposed on the top surface 132 of the second substrate 130. Once the adhesive is applied to one of the surfaces 122, 132, the two substrates 118, 130 can be aligned, mated or otherwise placed in contact with each other. After the adhesive cures, which may involve heating or exposing the adhesive to light depending on the exact adhesive selected, the two substrates 118, 130 are bonded together, and the fluid flow paths 112, e.g., channels, are formed in the regions where no adhesive was disposed on either substrate 118, 130. The layer of adhesive may have any predetermined thickness and pattern in order to create channels 112 with desired dimensions and shapes. Furthermore, additional patterns and reference features, such as, arrows, logos 182 or written instructions can also be included in the adhesive layer to facilitate ease of use.

Referring now to FIG. 9A, the flow cell 110 may further include a frame 136. The frame 136 has an inside edge 140 and an outside edge 142, and the frame 136 defines a rectangular shaped opening 138. The frame 136 includes a recess 144 formed along the inside edge 140 for receiving and selectively securing (with, for example, an adhesive) the substrates 118, 130 to the frame 136. The frame 136 also includes a recess 172 formed around the outside perimeter near the outside edge 142. This recess 172 can optionally receive a gasket or compressible tubing to improve the seal when the flow cell 110 is being used in operation.

In certain embodiments, FIG. 9D shows a fully assembled flow cell 110 ready to be loaded by a user. As shown, the flow cell 110 has gaskets 115, 117 in place surrounding the inlet holes 114 and outlet holes 116. The gaskets 115, 117 can be placed in recesses or can be placed on a flat top surface 120 depending on the desired application. The flow cell 110 also has a compressible tube 190 disposed in the recess 172. In an alternative embodiments, an elastomeric material may be deposited around at least portions of the periphery and be effective to substantially seal the device to avoid or reduce the likelihood of fluid loss or leakage.

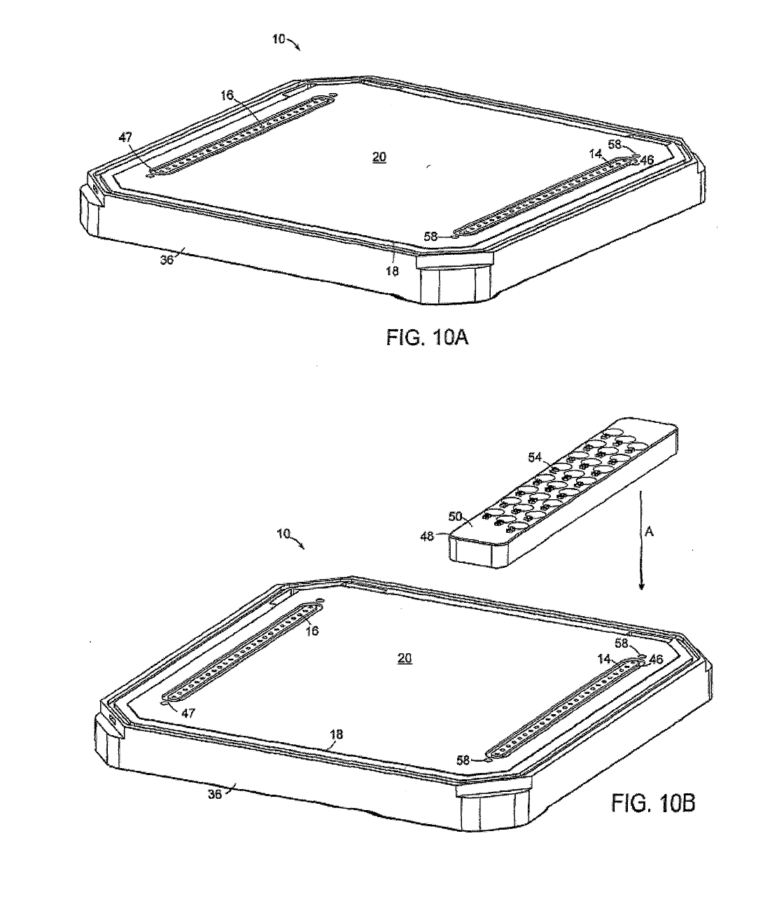

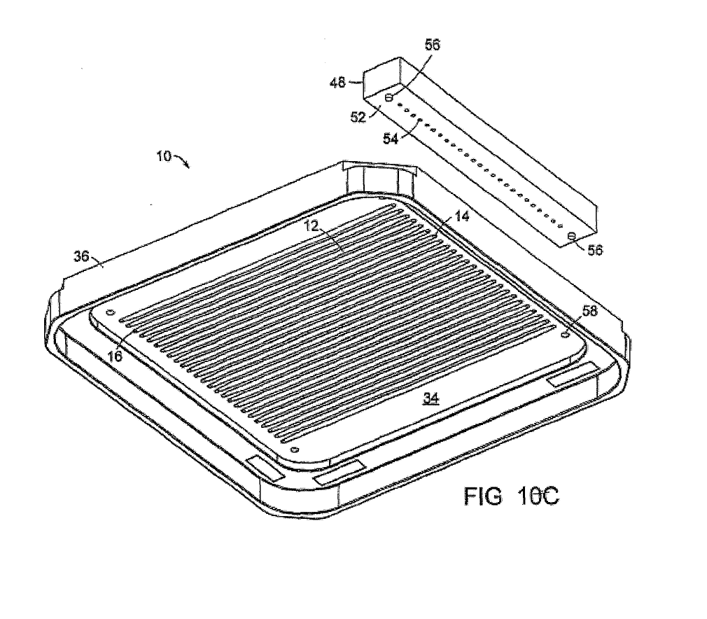

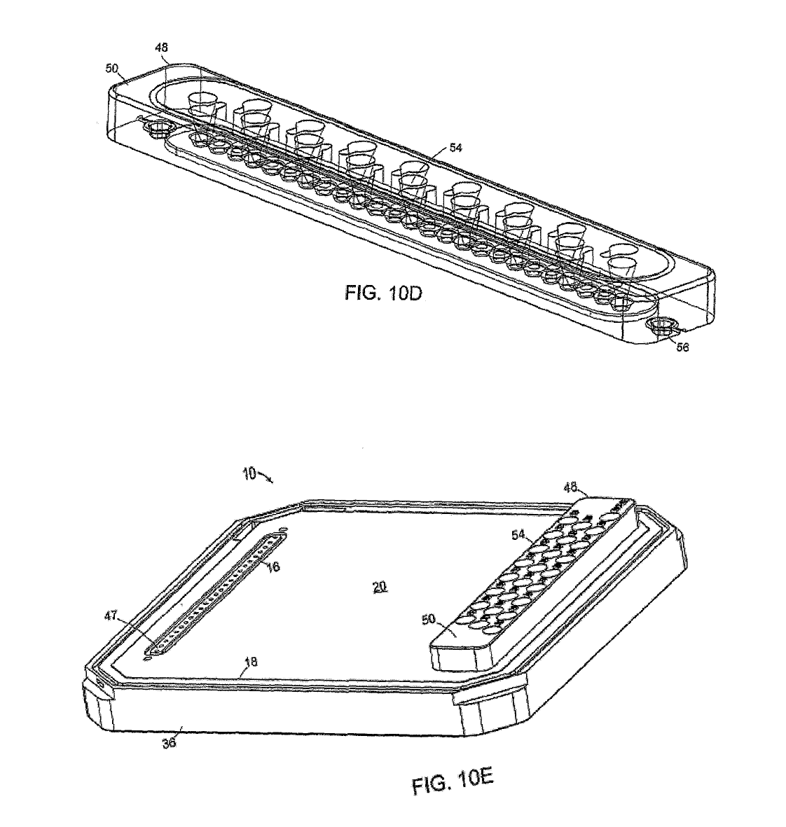

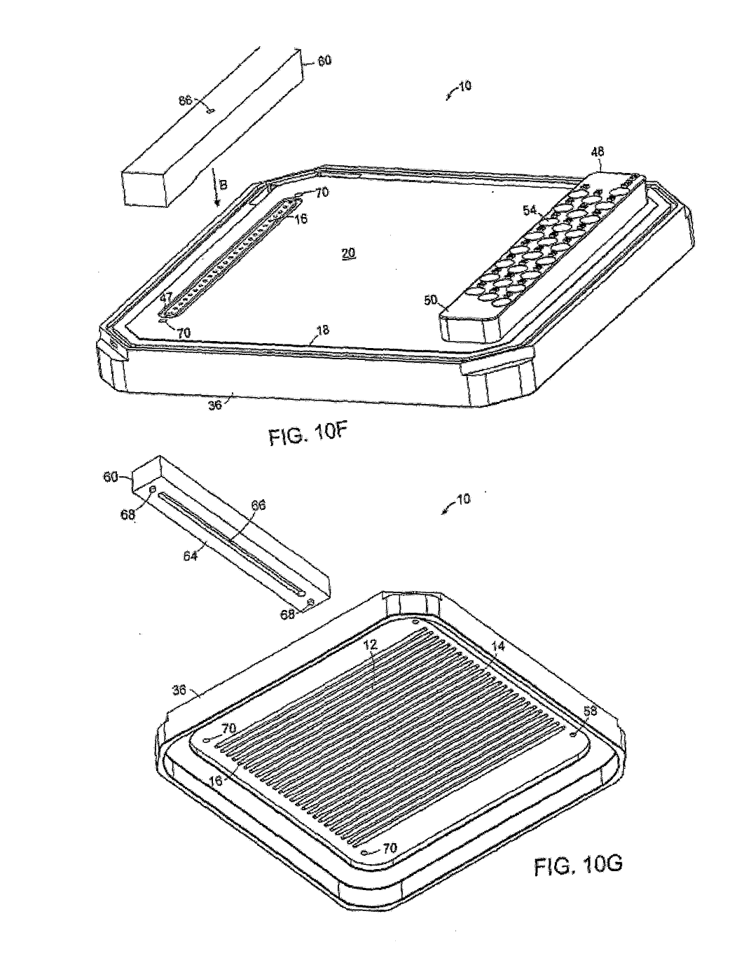

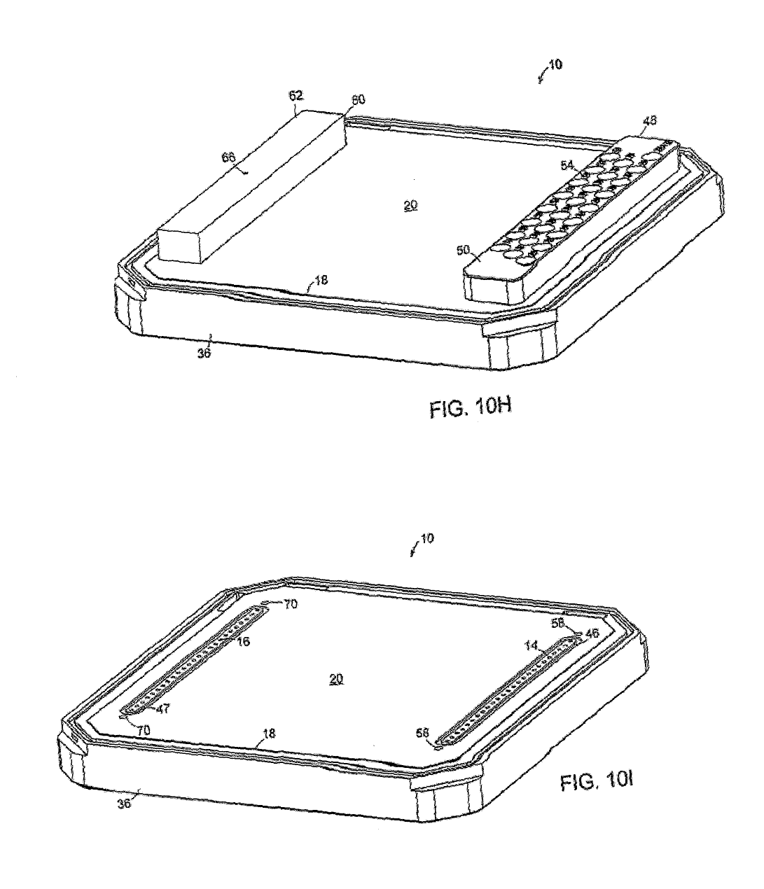

Referring now to FIGS. 10A-10H, wherein the process of loading samples into the flow cell 10 is shown and described. Referring now to FIG. 10A, there is shown a fully assembled flow cell 10. Although not shown in this particular illustration, each inlet hole 14 can be fluidically coupled to a channel 12, which is fluidically coupled to an outlet hole 16, such that when a fluid is loaded into the inlet hole 14, it can flow through the channel 12 and then be removed by the outlet hole 16. Optionally, a recessed canal 46 can be formed in the top surface 20 of the first substrate 18 completely surrounding the inlet holes 14. An additional recessed canal 47 can be formed in the top surface 20 of the first substrate 18 completely surrounding the outlet holes 16. These canals 46, 47 can optionally receive a gasket or compressible tubing for sealing the inlet holes 14 and outlet holes 16 during the processes of loading and unloading of the channels 12 and when the flow cell 10 is being used in operation. In the alternative, an elastomeric material may be deposited around at least portions of the periphery and be effective to substantially seal the device to avoid or reduce the likelihood of fluid loss or leakage.

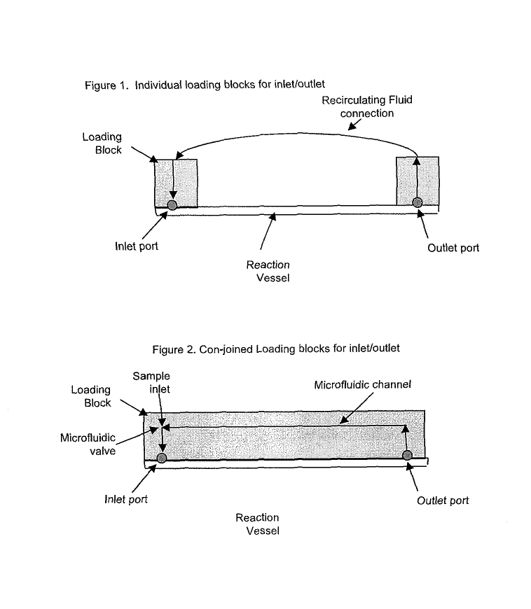

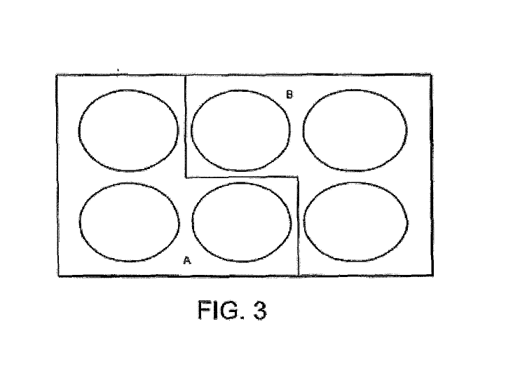

Referring now to FIGS. 10B-10E, a loading block 48 is provided for loading fluids into the channels 12 of the flow cell 10 for analysis. The loading block 48 has a top surface 50 and a bottom surface 52. The loading block 48 includes a plurality of loading wells 54 that extend through the loading block 48 from the top surface 50 to the bottom surface 52. As shown, the loading wells 54 can be substantially conically shaped with the widest diameter near the top surface 50 and the narrowest diameter near the bottom surface 52. At the top surface 50, the loading wells 54 can be arranged, for example, in three staggered rows. The loading wells 54 then angle toward the center of the loading block 48 forming a single row (FIG. 10C) at the bottom surface 52, such that the loading wells 54 align with the inlet holes 14. Alternatively, as shown in FIG. 10D, the loading wells 54 in the center row can be circular at the top surface 50 and the two outside rows can have a keyhole shape at the top surface 50. The loading wells 54 then taper down to a single row at the bottom surface 52 of the loading block 48. The loading wells 54 can be any shape or size necessary to facilitate loading a sufficient amount of sample into the channels 12 of the flow cell 10. The loading block may be used by itself or with additional loading blocks as shown, for example, in FIGS. 1 and 2. The loading blocks may be separate or may be in fluid communication, at least some portion, to facilitate loading of a sample.

In certain examples, the loading block 48 can further include two raised features known as mating pins 56 protruding from the bottom surface 52. The mating pins 56 align with, and are received into receive holes 58 in the top surface 20 of the first substrate 18. When the loading block 48 is lowered onto the flow cell 10 in the direction indicated by line A on FIG. 10B, the mating pins 56 are inserted into the receiving holes 58. This ensures proper alignment of the loading wells 54 and the inlet holes 14, such that fluid can flow from the loading wells 54, into the inlet hole 14 and then into the channel 12. A gasket or compressible tubing (not shown) may be installed in the canal 46 to provide a tight seal around the inlet holes 14 so that during the loading process, the fluid is contained in the loading block 48 and channels 12 and does not leak onto other areas of the flow cell 10. Alternatively, the loading block 48 can be made from a relatively soft, elastomeric material (e.g., silicone rubber, natural rubber, a fluoropolymer or other suitable materials) with additional raised features on the bottom surface 52 around each loading well 54 so that the loading block 48 itself forms the tight seal without a gasket. The additional raised features on the loading block 48 ensure an effective seal between the loading wells 54 and the top surface 20. These raised features are ridges which can either be rectangular or hemi-circular in cross section in order to provide the correct sealing geometry. FIG. 10E illustrates the loading block 48 in position on the flow cell 10.

Referring now to FIGS. 10E-10H, an unloading block 60 is provided for removing fluids from the channels 12. The unloading block 60 has a top surface 62 and a bottom surface 64. Referring in particular to FIG. 10F, the unloading block 60 includes a single aperture 66, extending through the unloading block 60 from the top surface 62 to the bottom surface 64. As shown in FIGS. 10F and 10G, the aperture 66 is cylindrical near the top surface 62, and a groove at the bottom surface 64. However, it will we apparent to one skilled in the art, given the benefit of this disclosure, that other shapes and sizes of apertures may be formed in the unloading block 60 for removing fluid from the channels 12. Examples include, a plurality of holes or a single duct extending though the unloading block 60. In an alternative embodiment, the block or manifold that connects each channel 12 to one or more adjacent channels can be etched or machined directly into the glass of the flow cell. In one embodiment, the unloading block 60 and/or the interface between the unloading block 60 and the outlet hole 16 is designed such that when the vacuum is applied during evacuation of the channels 12 (or while processing samples), there is a substantially uniform pressure distribution across all of the outlet holes 16. The substantially uniform pressure distribution equalizes the flow rates of samples and reagents in the channels 12. In some examples, the flow rate within the channels may vary by about 5% or less, 4% or less, 3% or less, 2% or less or 1% or less and still be considered substantially uniform. An optional surface treatment can be added to the channels to make them hydrophobic or hydrophilic in order to control the flow and to prevent it from moving from channel to channel.

In some examples, the unloading block 60 may further include two or more external projections, bosses or protrusions, referred to in certain instances as mating pins 68 protruding from the bottom surface 64. The mating pins 68 mate or otherwise align with, and are received into, a concave surface such as, for example, receiving holes 70 in the top surface 20 of the first substrate 18. When the unloading block 60 is lowered onto the flow cell 10 in the direction indicated by line B on FIG. 10F, the mating pins 68 are inserted into the receiving holes 70. This mating ensures proper alignment of the aperture 66 and the outlet holes 16, such that fluid can flow from the channels 12, through the outlet holes 16 and out of the flow cell 10 through the aperture 66. A gasket or compressible tubing (not shown) installed in the canal 47 provides a tight seal around the inlet holes 14 so that during the unloading process, the fluid is contained in the unloading block 60 and does not leak onto other areas of the flow cell 10. As with the loading block 48, the unloading block 60 can be made from a relatively soft, elastomeric material (e.g., silicone rubber, natural rubber, a fluoropolymer or other suitable materials) with additional raised features on the bottom surface 64 around the aperture 66 so that the unloading block 60 itself forms the tight seal without a gasket. The additional raised features on the unloading block 60 ensure an effective seal between the aperture and the top surface 20. These raised features are ridges which can either be rectangular or hemi-circular in cross section in order to provide the correct sealing geometry. FIG. 10H illustrates the flow cell 10 with the loading block 48 and unloading block 60 in position and ready for handling microfluidic volumes and related biological materials.

In operation, the user pre-loads the flow cell 10 with a buffer to hydrate or rehydrate the channels 12. This is accomplished by dispensing a microfluidic volume of buffer into the loading wells 54 either individually or simultaneously. This process may either be performed robotically or manually using a single pipette or a multi-gang pipette. Performing such an operation robotically is described is U.S. patent application Ser. No. 11/184,360, which is incorporated herein by reference. Once the buffer (or other liquid sample) is loaded, the buffer travels through the conical loading wells 54, down through the inlet hole 14, and then into the channels 12 via capillary action. After waiting a predetermined amount of time, the user attaches a vacuum pump, or other suitable device, to the unloading block 60, and pumps out the buffer from the flow cell 10.

In some examples, the user may then dispense a microfluidic volume of sample into the loading wells 54 either individually or simultaneously. As described above, this process may either be performed robotically, or manually using a single pipette or a multi-gang pipette. Once the sample is loaded, it travels through the conical loading wells 54, down through the inlet hole 14 and then into the channels 12 via capillary action. The user waits the appropriate amount of time for the samples to hybridize, and then pumps out the sample from the flow cell 10. Each loading well 54 and corresponding channel 12 may be isolated from the adjacent loading wells and channels, so that multiple distinct samples can be loaded and analyzed simultaneously without cross-contamination. This process of loading and unloading additional buffer solutions or reagents can be repeated as necessary for the particular analysis being performed. Once the flow cell 10 has been unloaded for the final time, the user detaches the vacuum pump from the unloading block 60, and then removes the loading block 48 and unloading block 60. As shown in FIG. 10I, the flow cell is now ready to be loaded into an apparatus for further analytical processes.

In various embodiments, the first substrate 18 or second substrate 30 can be treated to react with the microfluidic volumes being pulled through the flow cell 10. For example, a plurality of DNA strings can be adhered to surfaces of the channels 12 that are formed by the substrates 18, 30. Capture molecules or other materials may be present in the flow cell such that species flowed into the device can be retained, at least for some period, in the flow cell, through one or more of hydrophobic interactions, salt bridge formation, van der Waals' interactions, hydrogen bonding or even covalent bond formation.

One application for a flow cell 10 as described herein includes performing single molecule sequencing. In this application, the flow cell 10 includes individual strands of DNA or RNA (the “template”) bound to channels 12 of the flow cell 10. The template can be bound to the channels 12 by any of a variety of means for binding DNA or RNA to a surface using, for example, biotin-avidin interactions or other suitable attachment chemistries. In one example, the surface may include poly-T molecules covalently bound in the channels, and the RNA or DNA template may include a poly-A tail to hybridize to the poly-T molecule within the channel A primer is added that hybridizes to a portion of the DNA or RNA bound in the flow cell 10. Such an application is described in U.S. Publication No. 2006/0012784, filed Nov. 16, 2004 to Ulmer, which is incorporated herein by reference.

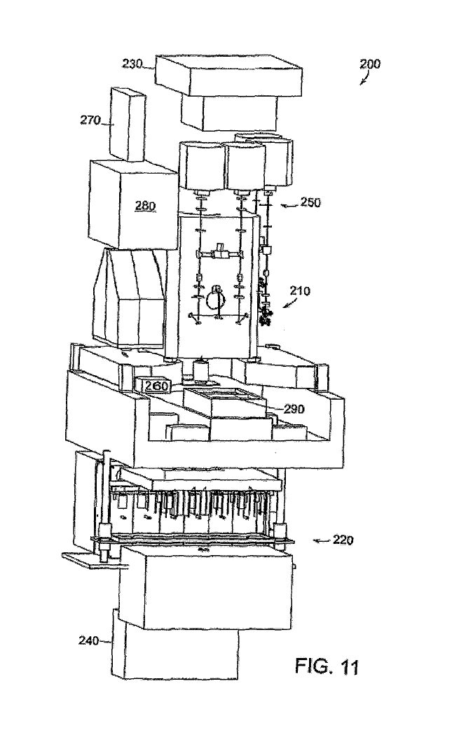

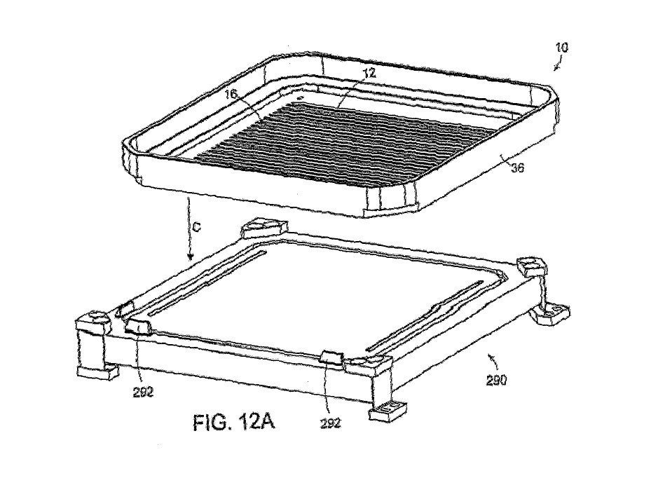

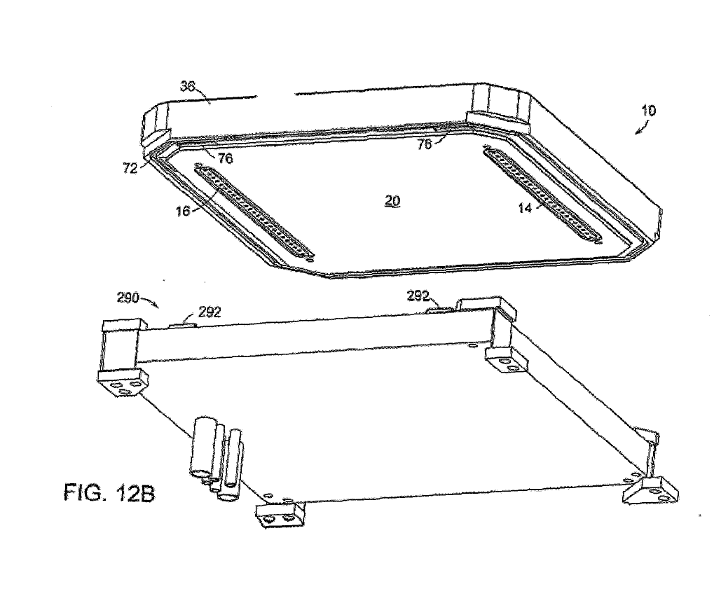

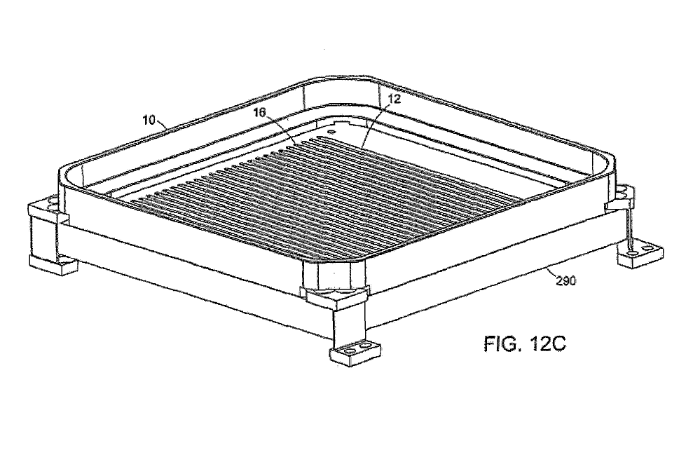

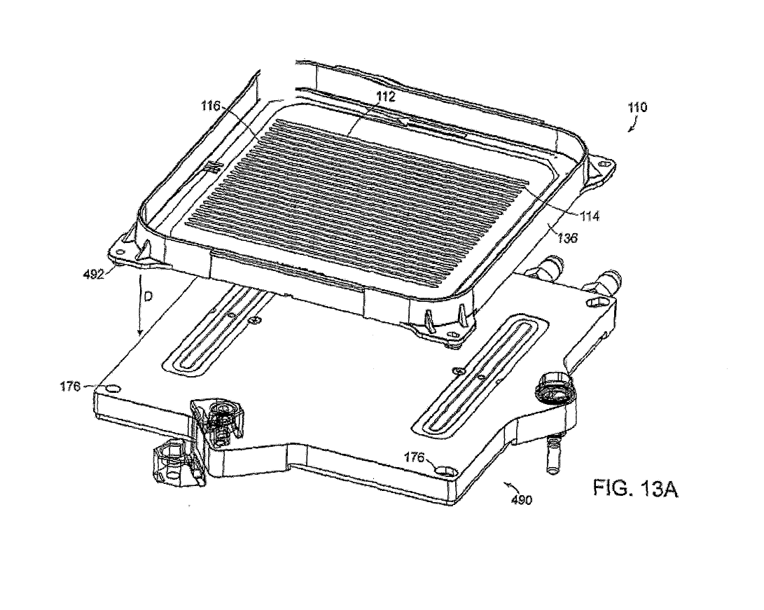

In certain embodiments, one example of an apparatus 200 that can be used to perform the processes described above is shown in FIG. 11. The apparatus 200 includes an optics section 210, a fluid handling section 220, a filter 230, a power supply 240, a laser control section 250, a bar code reader 260, a motor section 270, a central processing unit 280, and a flow chuck 290. After a flow cell, such as the flow cell 10, has been prepared for analysis, it may be loaded into the flow chuck 290 of the apparatus 200. Referring now to FIGS. 12A-12C, the flow cell 10 is being loaded into the flow chuck 290. The flow cell 10 is inverted by the user such that the top surface 20 of the first substrate 18 is placed in contact with the flow chuck 290 in the direction indicated by line C in FIG. 12A. The flow cell 10 optionally includes a recess 72 formed near the periphery of the frame 36 (FIG. 12B) and a gasket or compressible tube (not shown) may be received in the recess 72 to create a tighter seal when the flow cell 10 is installed in the flow chuck 290. The flow chuck 290 optionally includes posts 292 that are received into slots 76 in the flow cell 10. The posts 292 are alignment features designed to ensure the flow cell 10 is mounted into the flow chuck 290 correctly. FIG. 12C shows the flow cell 10 mounted in the flow chuck 290 and ready for processing by the apparatus 200.

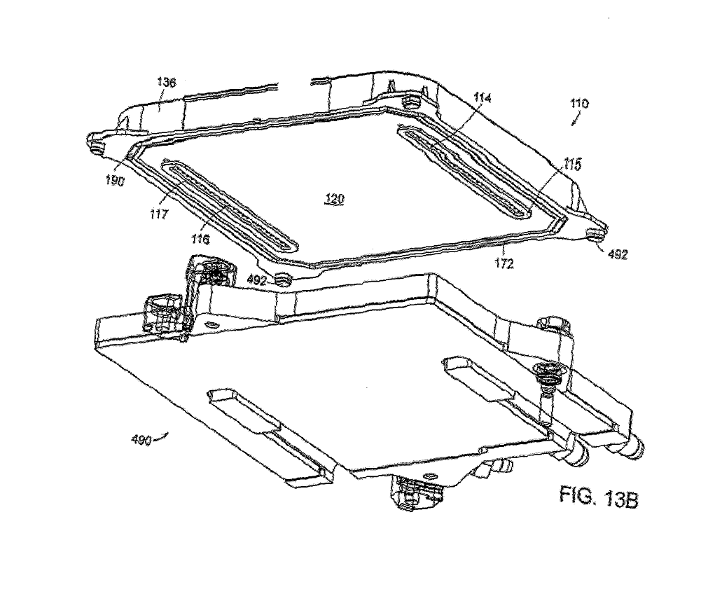

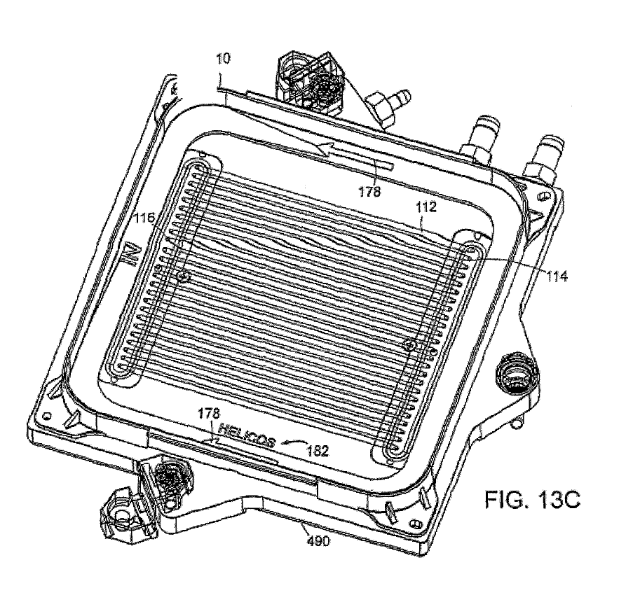

Referring now to FIGS. 13A-13C, the flow cell 110 is being loaded into another embodiment of a flow chuck 490. The flow cell 110 is inverted by the user such that the top surface 120 of the first substrate 118 is placed in contact with the flow chuck 490 in the direction indicated by line D in FIG. 13 A. As shown in FIG. 13B, the flow cell 110 has the compressible tube 190 disposed in the recess 172 to create a tighter seal when the flow cell 110 is installed in the flow chuck 490. In this embodiment, the flow cell 110 includes the posts 492 and the flow chuck 490 includes slots 176 to ensure proper positioning of the flow cell 110 in the flow chuck 490. The posts 492 also provide protection for the flow cell 110 so that the substrates 118, 130 do not break, crack or fracture if accidentally dropped or put down improperly on the flow chuck 490. Additional alignment features of this embodiment of the flow cell 110 include arrows 178 and an optional logo 182. FIG. 13C shows the flow cell 110 mounted in the flow chuck 490 and ready for processing by the apparatus 200. Alternate embodiments of the flow cell may also include bar coding or other electromagnetic devices to ensure proper loading and to identify samples that are being analyzed.

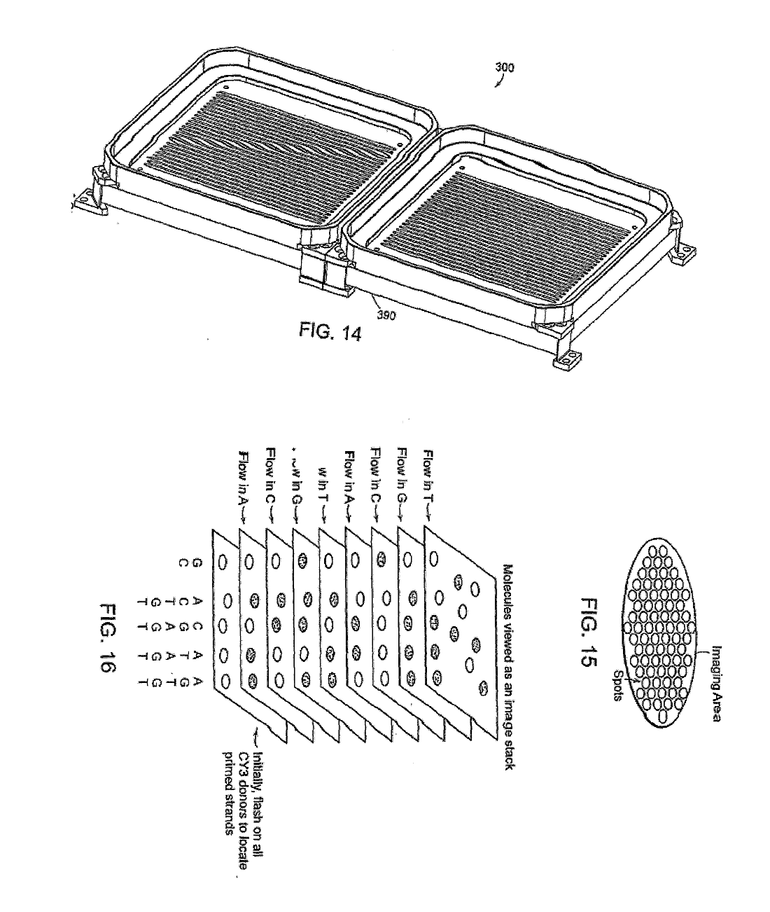

In certain embodiments, flow cells may be used individually, or optionally two or more flow cells may be combined together to analyze even more samples simultaneously. For example, FIG. 14 illustrates a dual flow cell 300, dual flow chuck 390 configuration. Although certain embodiments have been described, such description is for illustrative purposes only. Changes and variations may be made and are within the scope of this disclosure.

In certain examples, the components described herein, e.g., loading blocks and flow cells may be used in performing a variety of biochemical assays. In one embodiment, the biochemical assay comprises a sequencing-by-synthesis process. In another embodiment, sequencing-by-synthesis is conducted on single, optically-isolated nucleic acid duplexes attached to a surface. Certain methods combine the reaction component of sequencing-by-synthesis in parallel with effective imaging in order to sequence target nucleic acids of interest with high efficiency and high accuracy.

In the illustrative embodiments described below, sequencing-by-synthesis is used as the exemplary biochemical assay. However, the flow cells described herein can be used for any biochemical assay that has a reaction component and a interrogation component, where the reaction and interrogation components are typically conducted in sequence in (or on) the same chamber.

In some examples, where the reaction time for the biochemical assay is about the same as the interrogation time, the methods described herein comprise using a flow cell having a first and second area as described above. Where the biochemical assay is a sequencing-by-synthesis process, one or more nucleic acid duplexes comprising a template and a primer hybridized thereto can be attached to a surface of a first imaging area of the flow cell. One or more nucleic acid duplexes comprising a template and a primer hybridized thereto can be attached to a surface of a second imaging area of the flow cell. The duplexes can include an optically-detectable label that is used to determine the position of individual duplexes on the surface. Once duplex positions are obtained, the reaction component (e.g., sequencing reaction) is performed on the first and second imaging areas of the flow cell. After completion of the sequencing reaction, the first imaging area is interrogated (e.g., imaged).

In certain embodiments, during this first round of the sequencing-by-synthesis process, the surfaces of both imaging areas are exposed to a labeled nucleotide triphosphate in the presence of a polymerase. Template strands that contain the complement of the labeled nucleotide immediately adjacent the 3′ terminus of the primer incorporate the added nucleotide. After a wash step to remove unincorporated nucleotide, the surface of the first imaging area is interrogated to determine which duplex positions have had a label added, those being the positions that have incorporated the added nucleotide, as described herein. While the first imaging area is being interrogated, the surface of the second imaging area can be stored in a suitable buffer to maintain the stability of the attached duplexes, for example in a neutral buffer such as a HEPES buffer or other suitable buffer.

In some embodiments, after interrogation of the surface of the first imaging area is completed, the surface of the second imaging area is interrogated in a similar fashion. The surface of the second imaging area can be washed after storage and before interrogation. While the surface of the second imaging area is being interrogated, the sequencing reaction is performed on the surface of the first imaging area as described above. After interrogation, the added label can be removed. The surface of the first imaging area can be stored in a neutral buffer, as described above, until it is time to interrogate the surface of the first imaging area again. After interrogation of the surface of the second imaging area is completed, the surface of the first imaging area is interrogated as described above. The surface of the first imaging area can be washed after storage and before interrogation. While the surface of the first imaging area is being interrogated, the sequencing reaction is performed on the surface of the second imaging area as described above. After interrogation, the added label can be removed. The surface of the second imaging area can be stored in a neutral buffer, as described above, until it is time to interrogate the surface of the second imaging area. In this manner, the reaction component and the interrogation component of the biochemical assay are performed in parallel using the same flow cell. This process may be repeated iteratively until a desired number of nucleotide bases are sequenced, e.g., 25, 30, 50, 100 or more bases may be sequenced in each imaging area.

In some examples, after a sufficient number of reactions have been performed, the data set produced is a stack of image data for each imaging area that shows the linear results of the reaction component of the biochemical assay. For example, where the biochemical assay is a sequencing-by-synthesis process, after a sufficient number of nucleotides (determined by the desired read length as discussed below) have been exposed to the surface-bound templates of the first and second imaging areas, the data set produced is a stack of image data for each imaging area that shows the linear sequence of the individual duplex positions identified on the surface of that imaging area.

In embodiments where the reaction time required of the biochemical assay is greater than the interrogation time, the flow cell comprises at least two imaging areas, each having a surface, wherein biological molecules of interest are attached in multiple spots on each surface. For example, where the biochemical assay is a sequencing-by-synthesis process, as described above, duplexes are attached to the surfaces of each imaging area such that each surface has two or more spots where the duplexes are attached.

In certain embodiments, the number of spots per imaging area will depend upon the ratio of the reaction time to the interrogation time. For example, if the sequencing reaction takes three times as long as the interrogation, then the duplexes can be attached to each surface in three spots. Each spot is interrogated separately. The total interrogation time per imaging area is generally the time it takes to interrogate each spot, multiplied by the number of spots per imaging area. The reaction time is generally the time it takes to perform the reaction component on one spot because they are processed simultaneously in the same imaging area. The time it takes to interrogate all of the spots in one imaging area will approximate the amount of time it takes to complete the sequencing reaction for the other imaging area. FIG. 15 shows an illustrative schematic of multiple spots in an imaging area.

Where the reaction time of the biochemical assay is less than the interrogation time, then the flow cell may include three or more imaging areas as described above. The method of using the flow cell comprising three or more imaging areas comprises attaching the biochemical molecules required for the particular biochemical assay to the surfaces of each of the imaging areas. For example, where the biochemical assay is a sequencing-by-synthesis process, duplexes as described above are attached to the surfaces of each of the imaging areas. Once duplex positions are obtained, the reaction component of the biochemical assay can be performed simultaneously on each of the imaging areas of the flow cell.

In one embodiment, the surfaces of the imaging areas are exposed to a labeled nucleotide triphosphate in the presence of a polymerase and optionally suitable cofactors, salts, buffers and the like to facilitate incorporation of the labeled nucleotide triphosphate. Template strands that contain the complement of the labeled nucleotide immediately adjacent the 3′ terminus of the primer incorporate the added nucleotide. After a wash step to remove unincorporated nucleotide, the surface of the first imaging area is interrogated in order to determine which duplex positions have a label added, those being the positions that have incorporated the added nucleotide. While the surface of the first imaging area is being interrogated, the surfaces of the other imaging areas can be maintained in a suitable buffer as described above. After interrogation of the surface of the first imaging area, the label can be removed.

In certain embodiments, the surface of the second imaging area may then be interrogated and the reaction component of the biochemical assay {e.g., the sequencing reaction) is performed on the surface of the first imaging area as described above. After a wash step to remove unincorporated nucleotide, the surface of the first imaging area is stored, as described above, until it is time to interrogate the surface of the first imaging area. The interrogation {e.g., imaging) of the second imaging area is performed in parallel with the reaction component {e.g., sequencing) of the first imaging area. After interrogation of the surface of the second imaging area, the label can be removed.

In additional embodiments, the surface of the third imaging area may then be interrogated and the reaction component of the biochemical assay {e.g., the sequencing reaction) is performed on the surface of the second imaging area as described above. After a wash step to remove unincorporated nucleotide, the surface of the second imaging area is stored, as described above, until it is time to interrogate the surface of the second imaging area. The interrogation {e.g., imaging) of the third imaging area is performed in parallel with the reaction component {e.g., sequencing) of the second imaging area. After interrogation of the surface of the third imaging area, the label can be removed.

In certain examples, the cycle of performing sequencing-by-synthesis and interrogation in parallel can be repeated. After a sufficient number of reactions have been performed the data set produced is a stack of image data for each imaging area that shows the linear results of the reaction component of the biochemical assay. For example, where the biochemical assay is a sequencing-by-synthesis process, after a sufficient number of nucleotides (determined by the desired read length as discussed below) have been exposed to the surface-bound templates of the imaging areas, the data set produced is a stack of image data for each imaging area that shows the linear sequence of nucleotides incorporated at each of the individual duplex positions identified on the surface of that imaging area.

In certain embodiments, the number of imaging areas can be increased, depending on the ratio of reaction time to interrogation time. Generally, the number of imaging areas can be the same as the fold difference between reaction time and interrogation time. For example, if the reaction takes twice as long as the interrogation, then the flow cell can comprise two imaging areas. If the reaction takes three times as long, then the flow cell can comprise three imaging areas; five imaging areas for a five-fold difference, ten imaging areas for a ten-fold difference, twenty imaging areas for a twenty-fold difference, and so on.

In certain examples, the methods described herein can be used to provide de novo sequencing, re-sequencing, DNA fingerprinting, polymorphism identification, for example single nucleotide polymorphisms (SNP) detection, as well as applications for genetic cancer research. Applied to RNA sequences, the methods are useful to identify alternate splice sites, enumerate copy number, measure gene expression, identify unknown RNA molecules present in cells at low copy number, annotate genomes by determining which sequences are actually transcribed, determine phylogenic relationships, elucidate differentiation of cells, and facilitate tissue engineering. The methods may also be used to analyze activities of other biomacromolecules such as RNA translation and protein assembly.

In certain embodiments, methods for single molecule sequencing of nucleic acid templates comprise conducting a template-dependent sequencing reaction in which multiple labeled nucleotides are incorporated consecutively into a primer such that the accuracy of the resulting sequence is at least 70% with respect to a reference sequence. The primer is part of an optically-isolated substrate-bound duplex comprising a nucleic acid template having the primer hybridized thereto. The duplex is bound to the substrate such that the duplex is individually optically resolvable on the substrate. As described herein, a plurality of labeled nucleotides are incorporated consecutively into one or more individual primer molecules. In some embodiments, at least three consecutive nucleotides, each comprising an optically-detectable label, are incorporated into an individual primer molecule. In other embodiments, at least 5, at least 10, at least 20, at least 30, at least 50, at least 100, at least 500, at least 1000 or at least 10000 consecutive nucleotides, each comprising an optically-detectable label, are incorporated into an individual primer molecule.

In certain examples, the accuracy of the resulting sequence is at least about 70% with respect to a reference sequence, between about 75% and about 90% with respect to a reference sequence, or between about 90% and about 99% with respect to a reference sequence. In some examples, the accuracy of the resulting sequence can be greater than about 99% with respect to a reference sequence. The reference sequence can be, for example, the sequence of the template nucleic acid molecule, if known, or the sequence of the template obtained by other sequencing methods, or the sequence of the a corresponding nucleic acid from a different source, for example from a different individual of the same species or the same gene from a different species.

In some examples, methods for single molecule nucleic acid sequencing also comprise incorporating at least three consecutive nucleotides, each comprising an optically-detectable label, into a primer. The primer is part of a template/primer duplex. The template, primer or both is/are attached to a solid substrate such that the duplex is individually optically resolvable.

In one embodiment, all four nucleotides are added during the biochemical component of each cycle, with each nucleotide containing a detectable label. In some embodiments, the label attached to added nucleotides is a fluorescent label. Examples of fluorescent labels include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′ 5″-dibromopyrogallol-sulfonaphthalein (Bromopyrogallol Red); 7-diethyl amino-3-(4′-isothiocyanatophenyl)-4-methylcoumarin; diethylenetriamine pentaacetate; 4,4′-diisothiocyanatodihydro-stilbene-2,2-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. Some particularly desirable labels are cyanine-3 and cyanine-5. Labels other than fluorescent labels are contemplated by the invention, including other optically-detectable labels.

In certain embodiments, a surface for the imaging areas of the flow cells is an epoxide surface on a glass or fused silica slide or cover slip. However, any surface that has low native fluorescence is useful in the invention. Other surfaces include, but are not limited to, polytetrafluoroethylene, polyelectrolyte multilayers, and other materials that are substantially optically transparent at a desired excitation and/or emission wavelength. It is desirable that a surface have both low native fluorescence and have the ability to bind nucleic acids, either directly or indirectly.

In some embodiments, nucleic acid template molecules are attached to a substrate (also referred to herein as a surface) and subjected to analysis by single molecule sequencing as described herein. Nucleic acid template molecules are directly or indirectly (e.g., via a polymerase) attached to the surface such that the template/primer duplexes are individually optically resolvable. Substrates for use can be two- or three-dimensional and can comprise a planar surface (e.g., a glass slide) or can be shaped. A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites. The surface may also comprise protein or solid state nanopores, typically for non-optical detection as described below.

In certain examples, suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a nucleic acid. Substrates can include planar arrays or matrices capable of having regions that include populations of template nucleic acids or primers. Examples include nucleoside-derivatized CPG and polystyrene slides; derivatized magnetic slides; polystyrene grafted with polyethylene glycol, and the like.

In one embodiment, a substrate can be coated to allow optimum optical processing and nucleic acid attachment. Substrates for use can also be treated to reduce background. Illustrative coatings include epoxides, and derivatized epoxides (e.g., with a binding molecule, such as streptavidin). The surface can also be treated to improve the positioning of attached nucleic acids (e.g., nucleic acid template molecules, primers, or template molecule/primer duplexes) for analysis. As such, a surface can be treated with one or more charge layers (e.g., a negative charge) to repel a charged molecule (e.g., a negatively charged labeled nucleotide). For example, a substrate can be treated with polyallylamine followed by polyacrylic acid to form a polyelectrolyte multilayer. The carboxyl groups of the polyacrylic acid layer are negatively charged and thus repel negatively charged labeled nucleotides, improving the positioning of the label for detection. Coatings or films applied to the substrate should be able to withstand subsequent treatment steps (e.g., photoexposure, boiling, baking, soaking in warm detergent-containing liquids, and the like) without substantial degradation or disassociation from the substrate.

Examples of substrate coatings include, vapor phase coatings of 3-aminopropyltrimethoxysilane, as applied to glass slide products, for example, from Molecular Dynamics, Sunnyvale, Calif. In addition, generally, hydrophobic substrate coatings and films aid in the uniform distribution of hydrophilic molecules on the substrate surfaces. In those embodiments of the that employ substrate coatings or films, the coatings or films that are substantially non-interfering with primer extension and detection steps are desirable. Additionally, it is also desirable that any coatings or films applied to the substrates either increase template molecule binding to the substrate or, at least, do not substantially impair template binding.

In certain embodiments, various methods can be used to anchor or immobilize the nucleic acid template molecule to the surface of the substrate. The immobilization can be achieved through direct or indirect bonding to the surface. The bonding can be by covalent linkage. See, Joos et al, Analytical Biochemistry 247:96-101, 1997; Oroskar et al, Clin. Chem. 42:1547-1555, 1996; and Khandjian, Mol. Bio. Rep. 11: 107-115, 1986. One method includes direct amine bonding of a terminal nucleotide of the template or the primer to an epoxide integrated on the surface. The bonding also can be through non-covalent linkage. For example, biotin-streptavidin (Taylor et al, J. Phys. D. Appl. Phys. 24:1443, 1991) and digoxigenin with anti-digoxigenin (Smith et al, Science 253:1122, 1992) are common tools for anchoring nucleic acids to surfaces and parallels. Alternatively, the attachment can be achieved by anchoring a hydrophobic chain into a lipid monolayer or bilayer. Other methods for known in the art for attaching nucleic acid molecules to substrates also can be used.

In certain examples, any polymerizing enzyme may be used with the methods and devices described herein. A preferred polymerase is Klenow with reduced exonuclease activity. Nucleic acid polymerases generally useful include DNA polymerases, RNA polymerases, reverse transcriptases, and mutant or altered forms of any of the foregoing. DNA polymerases and their properties are described in detail in, among other places, DNA Replication 2nd edition, Romberg and Baker, W.H. Freeman, New York, N.Y. (1991). Known conventional DNA polymerases that can be used include, but are not limited to, Pyrococcus furiosus (Pfu) DNA polymerase (Lundberg et al., 1991, Gene, 108: 1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent™ DNA polymerase, Cariello et al., 1991, Polynucleotides Res, 19: 4193, New England Biolabs), 9 [deg.] Nm™ DNA polymerase (New England Biolabs), Stoffel fragment, ThermoSequenase<(R)> (Amersham Pharmacia Biotech UK), Therminator™ (New England Biolabs), Thermotoga maritima (Tma) DNA polymerase (Diaz and Sabino, 1998 Braz J. Med. Res, 31:1239), Thermus aquaticus (Taq) DNA polymerase (Chien et al., 1976, J. Bacteoriol, 127: 1550), DNA polymerase, Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerase (also referred as Deep Vent™ DNA polymerase, Juncosa-Ginesta et al., 1994, Biotechniques, 16:820, New England Biolabs), UlTma DNA polymerase (from thermophile Thermotoga maritima; Diaz and Sabino, 1998 Braz J. Med. Res, 31:1239; PE Applied Biosystems), Tgo DNA polymerase (from thermococcus gorgonarius, Roche Molecular Biochemicals), E. coli DNA polymerase I (Lecomte and Doubleday, 1983, Polynucleotides Res. 11: 7505), T7 DNA polymerase (Nordstrom et al., 1981, J. Biol. Chem. 256:3112), and archaeal DPII/DP2 DNA polymerase II (Cann et al., 1998, Proc Natl Acad. Sci. USA 95:14250->5). Other DNA polymerases include, but are not limited to, ThermoSequenase<(R)>, 9 [deg.] Nm™, Therminator™, Taq, Tne, Tma, PfU, Tfl, Tth, TIi, Stoffel fragment, Vent™ and Deep Vent™ DNA polymerase, KOD DNA polymerase, Tgo, JDF-3, and mutants, variants and derivatives thereof. Reverse transcriptases useful in the invention include, but are not limited to, reverse transcriptases from HIV, HTLV-I, HTLV-II, FeLV, FIV, SIV, AMV, MMTV, MoMuLV and other retroviruses (see Levin, Cell 88:5-8 (1997); Verma, Biochim Biophys Acta. 473:1-38 (1977); Wu et al., CRC Crit Rev Biochem. 3:289-347 (1975)).

In certain embodiments, direct amine attachment is used to attach primer, template, or both as duplex to an epoxide surface. The primer or the template comprises an optically-detectable label in order to determine the location of duplex on the surface. At least a portion of the duplex is optically resolvable from other duplexes on the surface. The surface is preferably passivated with a reagent that occupies portions of the surface that might, absent passivation, fluoresce. Passivation reagents include amines, phosphate, water, sulfates, detergents, and other reagents that reduce native or accumulating surface fluorescence. Sequencing is then accomplished by presenting one or more labeled nucleotide in the presence of a polymerase under conditions that promote complementary base incorporation in the primer. One base at a time (per cycle) can be added and all bases have the same label. There can be a wash step after each incorporation cycle, and the label is either neutralized without removal or removed from incorporated nucleotides. After the completion of a predetermined number of cycles of base addition, the linear sequence data for each individual duplex is compiled. Numerous algorithms are available for sequence compilation and alignment as discussed below.

In resequencing, an embodiment for sequence alignment compares sequences obtained to a database of reference sequences of the same length, or within one or two bases of the same length, from the target in a look-up table format. In one embodiment, the look-up table contains exact matches with respect to the reference sequence and sequences of the prescribed length or lengths that have one or two errors (e.g., 9-mers with all possible 1-base or 2-base errors). The obtained sequences are then matched to the sequences on the lookup table and given a score that reflects the uniqueness of the match to sequence(s) in the table. The obtained sequences are then aligned to the reference sequence based upon the position at which the obtained sequence best matches a portion of the reference sequence. An illustration of the alignment process is provided below in the Example.

In another embodiment, fluorescence resonance energy transfer (FRET) can be used to generate signal from incorporated nucleotides in single molecule sequencing of the invention. FRET can be conducted, for example, as described in Braslaysky, et al., 100 PNAS: 3960-64 (2003), incorporated by reference herein. In one embodiment, a donor fluorophore is attached to the primer portion of the duplex and an acceptor fluorophore is attached to a nucleotide to be incorporated. In other embodiments, donors are attached to the template, the polymerase, or the substrate in proximity to a duplex. In any case, upon incorporation, excitation of the donor produces a detectable signal in the acceptor to indicate incorporation.

In another embodiment, nucleotides presented to the surface for incorporation into a surface-bound duplex comprise a reversible blocker. A preferred blocker is attached to the 3′-hydroxyl on the sugar moiety of the nucleotide. For example an ethyl cyanine (—OH—CH₂CH₂C═N) blocker, which can be removed by hydroxyl addition to the sample, is a useful removable blocker. Other useful blockers include fluorophores placed at the 3′-hydroxyl position, and chemically labile groups that are removable, leaving an intact hydroxyl for addition of the next nucleotide, but that inhibit further polymerization before removal.

In an additional embodiment, individually optically resolvable complexes comprising polymerase and a target nucleic acid are oriented with respect to each other for complementary base addition in a zero mode waveguide. In one embodiment, an array of zero-mode waveguides comprising sub-wavelength holes in a metal film is used to sequence DNA or RNA at the single molecule level. A zero-mode waveguide is one having a wavelength cut-off above which no propagating modes exist inside the waveguide. Illumination decays rapidly incident to the entrance to the waveguide, providing very small observation volumes. In one embodiment, the waveguide includes small holes in a thin metal film on a microscope slide or cover slip. Polymerase is immobilized in an array of zero-mode waveguides. The waveguide is exposed to a template/primer duplex, which is captured by the enzyme active site. Then a solution containing a species of fluorescently-labeled nucleotide is presented to the waveguide, and incorporation is observed after a wash step as a burst of fluorescence.

In certain embodiments, the exact detection method may be selected based, at least in part, on the particular type or label used. Exemplary detection methods include radioactive detection, optical absorbance detection, e.g., UV-visible absorbance detection, optical emission detection, e.g., fluorescence; phosphorescence or chemiluminescence; Raman scattering; non-optical methods such as, for example, detection using nanopores (e.g., protein or solid state) through which molecules are individually passed so as to allow identification of the molecules by noting characteristics or changes in various properties or effects such as capacitance or blockage current flow (see, for example, Stoddart et al, Proc. Nat. Acad. Sci., 106:7702, 2009; Purnell and Schmidt, ACS Nano, 3:2533, 2009; Branton et al, Nature Biotechnology, 26:1146, 2008; Polonsky et al, U.S. Application 2008/0187915; Mitchell & Howorka, Angew. Chem. Int. Ed. 47:5565, 2008; Borsenberger et al, J. Am. Chem. Soc., 131, 7530, 2009); or other suitable detection methods. For example, extended primers can be detected on a substrate by scanning all or portions of each substrate simultaneously or serially, depending on the scanning method used. For fluorescence labeling, selected regions on a substrate may be serially scanned one-by-one or row-by-row using a fluorescence microscope apparatus, such as described in Fodor (U.S. Pat. No. 5,445,934) and Mathies et al (U.S. Pat. No. 5,091,652). Devices capable of sensing fluorescence from a single molecule include scanning tunneling microscope (STM) and the atomic force microscope (AFM). Hybridization patterns may also be scanned using a CCD camera {e.g., Model TE/CCD512SF, Princeton Instruments, Trenton, N.J.) with suitable optics (Ploem, in Fluorescent and Luminescent Probes for Biological Activity Mason, T. G. Ed., Academic Press, Landon, pp. 1-11 (1993), such as described in Yershov et al, Proc. Natl. Acad. Sci. 93:4913 (1996), or may be imaged by TV monitoring. For radioactive signals, a phosphorimager device can be used (Johnston et al, Electrophoresis, 13:566, 1990; Drmanac et al., Electrophoresis, 13:566, 1992; 1993). Other commercial suppliers of imaging instruments include General Scanning Inc., (Watertown, Mass. on the World Wide Web at genscan.com), Genix Technologies (Waterloo, Ontario, Canada; on the World Wide Web at confocal.com), and Applied Precision Inc. Such detection methods are particularly useful to achieve simultaneous scanning of multiple attached template nucleic acids.

In certain examples, a number of approaches can be used to detect incorporation of fluorescently-labeled nucleotides into a single nucleic acid molecule. Optical setups include near-field scanning microscopy, far-field confocal microscopy, wide-field epi-illumination, light scattering, dark field microscopy, photoconversion, single and/or multiphoton excitation, spectral wavelength discrimination, fluorophore identification, evanescent wave illumination, and total internal reflection fluorescence (TIRF) microscopy. In general, certain methods involve detection of laser-activated fluorescence using a microscope equipped with a camera. Suitable photon detection systems include, but are not limited to, photodiodes and intensified CCD cameras. For example, an intensified charge couple device (ICCD) camera can be used. The use of an ICCD camera to image individual fluorescent dye molecules in a fluid near a surface provides numerous advantages. For example, with an ICCD optical setup, it is possible to acquire a sequence of images (movies) of fiuorophores.

Some embodiments described herein may use TIRF microscopy for two-dimensional imaging. TIRF microscopy uses totally internally reflected excitation light and is well known in the art. See, e.g., the World Wide Web at nikon-instruments.jp/eng/page/products/tirf.aspx. In certain embodiments, detection can be carried out using evanescent wave illumination and total internal reflection fluorescence microscopy. An evanescent light field can be set up at the surface, for example, to image fluorescently-labeled nucleic acid molecules. When a laser beam is totally reflected at the interface between a liquid and a solid substrate (e.g., a glass), the excitation light beam penetrates only a short distance into the liquid. The optical field does not end abruptly at the reflective interface, but its intensity falls off exponentially with distance. This surface electromagnetic field, called the “evanescent wave”, can selectively excite fluorescent molecules in the liquid near the interface. The thin evanescent optical field at the interface provides low background and facilitates the detection of single molecules with high signal-to-noise ratio at visible wavelengths.

In some examples, the evanescent field also can image fluorescently-labeled nucleotides upon their incorporation into the attached template/primer complex in the presence of a polymerase. Total internal reflectance fluorescence microscopy is then used to visualize the attached template/primer duplex and/or the incorporated nucleotides with single molecule resolution.

Nucleic acid template molecules include deoxyribonucleic acid (DNA) and/or ribonucleic acid (RNA). Nucleic acid template molecules can be isolated from a biological sample containing a variety of other components, such as proteins, lipids and non-template nucleic acids. Nucleic acid template molecules can be obtained from any cellular material, obtained from an animal, plant, bacterium, fungus, virus or any other organism. Nucleic acid template molecules may be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid. Nucleic acid template molecules may also be isolated from cultured cells, such as a primary cell culture or a cell line. The cells or tissues from which template nucleic acids are obtained can be infected with a virus or other intracellular pathogen. A sample can also be total RNA extracted from a biological specimen, a cDNA library, or genomic DNA.

In some examples, nucleic acid obtained from biological samples can be fragmented to produce suitable fragments for analysis. In one embodiment, nucleic acid from a biological sample is fragmented by sonication. Nucleic acid template molecules can be obtained as described in U.S. Patent Application 2002/0190663 A1, published Oct. 9, 2003, the teachings of which are incorporated herein in their entirety.

Generally, nucleic acid can be extracted from a biological sample by a variety of techniques such as those described by Maniatis, et al, Molecular Cloning: A Laboratory Manual, Cold Spring Harbor, N.Y., pp. 280-281 (1982). Generally, individual nucleic acid template molecules can be from about 5 bases to about 20 kb. Nucleic acid molecules may be single-stranded, double-stranded, or double-stranded with single-stranded regions (for example, stem- and loop-structures).

A biological sample as described herein may be homogenized or fractionated in the presence of a detergent or surfactant. The concentration of the detergent in the buffer may be about 0.05% to about 10.0%. The concentration of the detergent can be up to an amount where the detergent remains soluble in the solution. In a preferred embodiment, the concentration of the detergent is between 0.1% to about 2%. The detergent, particularly a mild one that is non-denaturing, can act to solubilize the sample. Detergents may be ionic or nonionic. Examples of non-ionic detergents include triton, such as the Triton® X series (Triton® X-100 t-Oct-C₆H₄—(OCH₂—CH₂)ₓOH, x=9-10, Triton® X-100®, Triton® X-114 x=7-8), octyl glucoside, polyoxyethylene(9)dodecyl ether, digitonin, IGEP AL(R) CA630 octylphenyl polyethylene glycol, n-octyl-beta-D-glucopyranoside (betaOG), n-dodecyl-beta, Tween® 20 polyethylene glycol sorbitan monolaurate, Tween® 80 polyethylene glycol sorbitan monooleate, polidocanol, n-dodecyl beta-D-maltoside (DDM), NP-40 nonylphenyl polyethylene glycol, C12E8 (octaethylene glycol n-dodecyl monoether), hexaethyleneglycol mono-n-tetradecyl ether (C14EO6), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl)dimethylammonio]-1-propanesulfonate. It is contemplated also that urea may be added with or without another detergent or surfactant.

Lysis or homogenization solutions may further contain other agents, such as reducing agents. Examples of such reducing agents include dithiothreitol (DTT), [beta]-mercaptoethanol, DTE, GSH, cysteine, cysteamine, tricarboxyethyl phosphine (TCEP), or salts of sulfurous acid.

Certain embodiments described herein provide advantages including, but not limited to, attachment of primer via a direct amine attachment to an epoxide surface. In some examples, a template may be attached first and have attached duplex (i.e., duplex was formed first and then attached to the surface). The surface may also be functionalized with one member of a binding pair, the other member of the binding pair being attached to the template, primer, or both for attachment to the surface. For example, the surface may be functionalized with stretptavidin, and biotin was attached to the termini of either the template, the primer, or both.

In certain embodiments, a method of facilitating loading of a flow cell is provided. In some examples, the method comprises providing at least a first and a second loading block comprising a fluidic coupling there between. In certain examples, at least one of, or both of, the first and second loading blocks may be constructed and arranged to couple to the flow cell to provide fluid to the flow cell, as described herein. In some examples, each of the loading blocks may include one or more of a microfluidic channel, a microfluidic valve, one or more inlets, one or more outlets and/or one or more fluid ports for fluidically coupling the first and second loading blocks.

Additional features, aspects and examples will be apparent to the person of ordinary skill in the art, given the benefit of this disclosure including, for example, and the specific examples described below

### EXAMPLES

### Example 1

In certain embodiments, sequencing may combine sample preparation, surface preparation and oligo attachment, interrogation, and analysis in order to achieve high-throughput sequence information. In one embodiment, optically-detectable labels may be attached to templates that are attached directly to an epoxide surface. Individual template molecules were imaged in order to establish their positions on the surface. Then, primer can be added to form duplex on the surfaces, and individual nucleotides containing an optical label were added in the presence of polymerase for incorporation into the 3′ end of the primer at a location in which the added nucleotide is complementary to the next-available nucleotide on the template immediately 5′ (on the template) of the 3′ terminus of the primer. Unbound nucleotide is washed out, scavenger is added, and the surface is imaged. Optical signal at a position previously noted to contain a single duplex (or primer) is counted as an incorporation event. Label is removed and the remaining linker is capped and the system is again washed. The cycle is repeated with the remaining nucleotides. A full-cycle is conducted as many times as necessary to complete sequencing of a desired length of template. Once the desired number of cycles is complete, the result is a stack of images as shown in FIG. 16 represented in a computer database. For each spot on the surface that contains an initial individual duplex, there will be a series of light and dark image coordinates, corresponding to whether a base was incorporated in any given cycle. For example, if the template sequence is TACGTACG and nucleotides were presented in the order CAGU(T), then the duplex would be “dark” (i.e., no detectable signal) for the first cycle (presentation of C), but would show signal in the second cycle (presentation of A, which is complementary to the first T in the template sequence). The same duplex would produce signal upon presentation of the G, as that nucleotide is complementary to the next available base in the template, C. Upon the next cycle (presentation of U), the duplex would be dark, as the next base in the template is G. Upon presentation of numerous cycles, the sequence of the template would be built up through the image stack. The sequencing data are then fed into an aligner as described below for resequencing, or are compiled for de novo sequencing as the linear order of nucleotides incorporated into the primer.

### Example 2

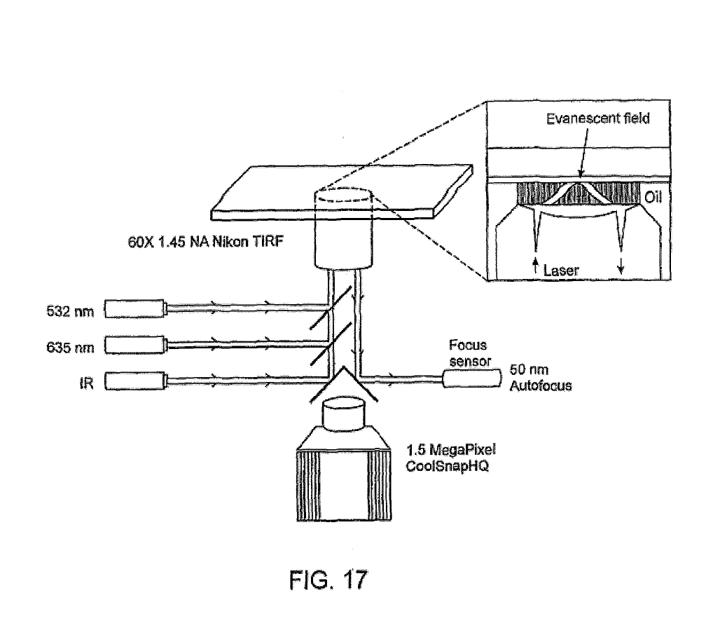

The imaging system to be used can be any system that provides sufficient illumination of the sequencing surface at a magnification such that single fluorescent molecules can be resolved. The imaging system used in this example described below is shown in FIG. 17. In general, the system comprises three lasers, one that produced “green” light, one that produces “red” light, and in infrared laser that aids in focusing. The beams are transmitted through a series of objectives and mirrors, and focused on the image as shown in FIG. 17. Imaging is accomplished with an inverted Nikon TE-2000 microscope equipped with a total internal reflection objective (Nikon). Alignment and/or compilation of sequence results obtained from the image stacks produced as generally described above utilizes look-up tables that take into account possible sequences changes (due, e.g., to errors, mutations, etc.). Sequencing results obtained as described herein were compared to a look-up type table that contains all possible reference sequences plus 1 or 2 base errors.

### Example 3

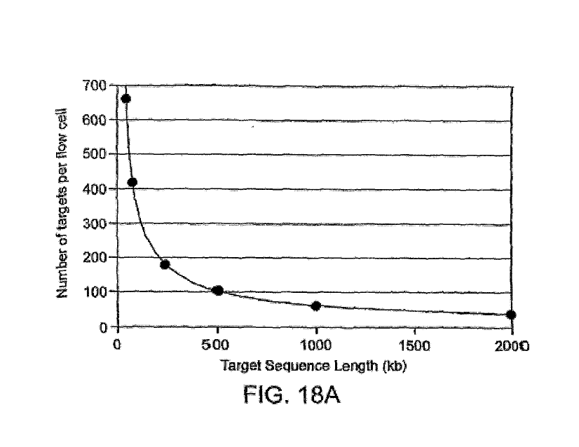

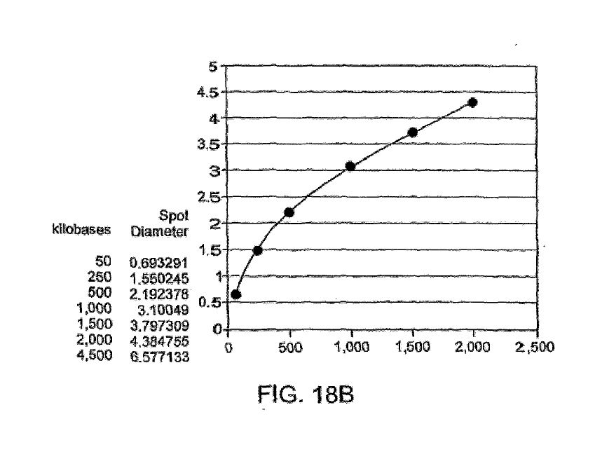

A graph may be constructed to determine the relationship between the number of targets per flow cell and the sequence length of the target nucleic acid of interest. One such graph is shown in FIG. 18A. As can be seen in the graph, as the length of the target nucleic acid of interest increases, the number of targets per flow cell generally decreases.

A graph of spot diameter versus kilobases of target nucleic acid of interest may also be created (see FIG. 18B). As can be seen in the graph, spot diameter typically increases as the size of the target nucleic acid increases.

When introducing elements of the examples disclosed herein, the articles “a,” “an,” “the” and “said” are intended to mean that there are one or more of the elements. The terms “comprising,” “including” and “having” are intended to be open-ended and mean that there may be additional elements other than the listed elements. It will be recognized by the person of ordinary skill in the art, given the benefit of this disclosure, that various components of the examples can be interchanged or substituted with various components in other examples.

All references cited herein are incorporated herein by reference in their entirety and for all purposes to the same extent as if each individual publication or patent or patent application was specifically and individually indicated to be incorporated by reference in its entirety for all purposes.

### EQUIVALENTS

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

1. A method for increasing recovery of an analyte in a sample, comprising providing a mechanism for sample recircularization in a flow cell device using a reactive vessel, such that the analyte in the sample has two or more exposures to the reactive vessel.

2. The method of claim 1, further comprising loading the sample in the flow cell device, said device comprising at least one inlet port and at least one outlet port, wherein each of the inlet and outlet ports is coupled to a loading block; and wherein the loading blocks are joined so as to permit sample loading or fluid recirculation through at least one reaction vessel.

3. The method of claim 2, further comprising providing at least a first and a second loading block comprising a fluidic coupling therebetween, in which at least one of the first and second loading blocks is constructed or arranged to couple to the flow cell to provide fluid to the flow cell.

4. A flow cell device comprising at least one inlet port and at least one outlet port, wherein each of the inlet and outlet ports is coupled to at least one loading block; and wherein the loading blocks are constructed and/or arranged, optionally including by joining, so as to permit sample loading or fluid recirculation through at least one reaction vessel.

5. The method of claim 3, wherein the loading blocks individually access each of the reaction vessels.

6. The method of claim 2, wherein the joining is by means of glass capillaries.

7. The method of claim 2, wherein after introducing sample, the flow cell device is closed permitting recirculation of the sample repeatedly through a reaction vessel.

8. The method of claim 2, wherein the recirculation is the result of temperature or electrical gradients.

9. The method of claim 2, wherein the reaction vessel is a channel.

10. The method of claim 2, wherein the inlet and outlet port access individual channels.

11. (canceled)

12. The method of claim 2, wherein the inlet and outlet port access individual reaction vessels.

13-15. (canceled)

16. The method of claim 2, wherein the reaction vessel includes a means of maintaining sample agitation by means of magnetic beads.

17. The method of claim 2, wherein the agitation is by means of fluid flow control back-forth.

18. The method of claim 2, wherein the reaction vessel has a single inlet and outlet and multiple reaction locations are defined by analytes specifically attached in defined locations.

19. (canceled)

20. The method of claim 18, wherein the analytes are synthesized at defined locations.

21. The method of claim 2, wherein the analytes in the samples are haptens, antibodies, or nucleic acids.

22. (canceled)

23. The method of claim 2, further comprising detecting and/or identifying samples using non-optical methods including nanopore detection.

24. A method for sequencing analysis of nucleic acid from individual cells, the method, comprising:
i. selecting individual cells;
ii. lysing of cells;
iii. capturing nucleic acids on surface;
iv. adding a universal sequence; and
v. sequencing at least a portion of the nucleic acid.

25. The method of claim 24, wherein the nucleic acids in individual cells are barcoded.

26. The method of claim 25, wherein the barcoding is via viral vectors or via transposons.

27. The method of claim 25, wherein the barcoding is via a spatial:temporal association.

28. (canceled)

29. The method of claim 24, wherein the surface is a microplate.

30. The method of claim 24, further comprising applying cells to the surface by direct mechanical spotting, inkjet spraying, or sonic spraying.

31. (canceled)

32. The method of claim 24, wherein the cells are red blood cells.

33. The method of claim 24, wherein the nucleic acids are fragmented prior to capture on the surface.

34. (canceled)

35. The method of claim 24, wherein the universal sequence is added via one or more of: ligation; a single dNTP and terminal deoxynucleotidyl transferase; or a single ATP and polyA polymerase.

36. The method of claim 24, wherein the surface is a bead, planar, or three dimensional.

37. (canceled)

38. The method of claim 37, wherein the capture oligonucleotides are 20-50 bases in length.

39. The method of claim 38, wherein the capture oligonucleotides comprise all possible combinations of the sequences found in the sample nucleic acid.

40. The method of claim 37, wherein the capture oligonucleotide has a sequence complementary to the universal primer.

41-42. (canceled)

43. The method of claim 24, wherein the sequencing is sequencing by synthesis, ligation or hybridization.

44. The method of claim 43, wherein multiple rounds of hybridization, detection, denaturing are performed each round using different interrogation oligonucleotides.

45. The method of claim 43, wherein the sequencing is on individual, optically resolvable molecules.

46. The method of claim 43, wherein the nucleic acid on the surface is amplified prior to sequencing.

47-58. (canceled)

59. The method of claim 24, wherein the universal sequence is added during a polymerase mediated copying of the nucleic acid.

60. The method of claim 59, wherein the copying additionally adds a functional group onto the copied fragments to enable chemical attachment to a surface.

61. The method of claim 60, wherein the functional group is an amine and the surface contains epoxides, or is a phosphate and enables ligation to surface anchored oligonucleotides.
